(19) United States
(12) Patent Application Publication
(10) Pub. No.: US 2021/0060108 A1
(43) Pub. Date: Mar. 4, 2021

(54) INULA HELENIUM EXTRACTS FOR TREATING AND PREVENTING POLLUTION RELATED DAMAGE

(71) Applicants: Fabien HAVAS, Bnei Dkalim (IL); Leah VON OPPEN-BEZALEL, Berlin (DE); Olga BEN-CHITRIT, Ashdod (IL); Inon PERRY, Tel Aviv (IL)

(72) Inventors: Fabien HAVAS, Bnei Dkalim (IL); Leah VON OPPEN-BEZALEL, Berlin (DE); Olga BEN-CHITRIT, Ashdod (IL); Inon PERRY, Tel Aviv (IL)

(21) Appl. No.: 16/957,814

(22) PCT Filed: Dec. 19, 2018
(86) PCT No.: PCT/IB2018/001555

§ 371 Date: Jun. 25, 2020

Related U.S. Application Data
(60) Provisional application No. 62/611,287, filed on Dec. 28, 2017.

(51) Int. Cl. A61K 36/28

(52) U.S. Cl. CPC A61K 36/28; A61K 2236/00

(57) ABSTRACT

Extracts derived from plants and/or fractions thereof, compositions comprising same and methods and use thereof in protecting tissues from damage caused by toxins are encompassed herein. More particularly, compositions comprising a polar solvent extract and/or a fraction thereof derived from an Inula plant (e.g., Inula helenium) and methods and use thereof in protecting tissues comprising epithelial cells from adverse effects of toxins, particularly exogenous toxins originating from environmental pollution, are encompassed herein.

### RELATED APPLICATIONS

This application claims priority of U.S. Provisional Application No. 62/611,287, filed Dec. 28, 2017, the entirety of which is incorporated herein by reference for all purposes.

### FIELD OF THE INVENTION

Extracts derived from plants and/or fractions thereof and compositions comprising same, as well as methods and use thereof for protecting tissues from damage caused by toxins (endogenous and exogenous toxins) are encompassed herein. In particular, compositions comprising a polar solvent extract and/or fraction thereof derived from Inula helenium and methods and use thereof for protecting tissues (e.g., tissues comprising epithelial cells) from adverse effects of exogenous toxins, particularly toxins originating from environmental pollution, are encompassed herein.

### BACKGROUND OF THE INVENTION

Epithelium is one of the four basic types of animal tissues, along with connective tissue, muscle tissue and nervous tissue. Epithelial tissues line surfaces and cavities throughout the body. Epithelium forms part of both the outside surface (skin) and the inside cavities and lumen of mammalian bodies. Functions of epithelial cells include protection, detection of sensation, selective absorption, secretion and transcellular transport. In that epithelial tissue comprises an interface between the body and the external environment, this tissue provides a defensive boundary against environmental toxins and thus, is particularly susceptible to adverse effects thereof.

### SUMMARY OF THE INVENTION

Extracts of Inula plants (e.g., Inula helenium) and compositions comprising same that are useful for protecting mammalian tissues from pollution and treating mammalian tissues that have been exposed to pollution and damaged thereby are described herein. The extracts and compositions described herein exhibit efficacy for preventing damage to tissues comprising epithelial cells following exposure to various pollutants, particularly chemical substances, including chemicals found in smoke (e.g., engine smoke, cigarette smoke and fire smoke) and in industrial waste. Furthermore, the extracts and compositions described herein exhibit therapeutic efficacy for treating tissues comprising epithelial cells damaged by exposure to various pollutants, such as chemical substances including chemicals found in smoke (e.g., engine smoke, cigarette smoke and fire smoke) and chemicals found in industrial waste.

The present invention is based in part on the unexpected discovery that extracts of Inula plants (e.g., Inula helenium) that are soluble in a polar solvent and fractions thereof are useful for protecting the skin, which serves as a representative of other epithelial-cell comprising tissues (e.g., lung tissue), from toxic challenges due to contact with exogenous pollutants. Extracts described herein also exhibit therapeutic efficacy when used to treat the skin and other epithelial-cell comprising tissues that have been damaged by exposure to contact with exogenous pollutants. Extracts described herein are also useful for restoring the appearance and vitality of skin damaged by exposure to contact with exogenous pollutants. Extracts and fractions thereof described herein, as well as compositions comprising same, also improve the appearance and vitality of skin and reduce premature skin aging. Extracts and fractions thereof described herein and compositions comprising same exhibit significant protective and therapeutic effects on cell viability, increasing the cell survival following exposure to a smoke-tainted medium.

According to one aspect, a method for protecting a tissue comprising epithelial cells from adverse effects of at least one pollutant is presented, the method comprising administering to a subject in need thereof a polar solvent extract derived from an Inula plant (e.g., Inula helenium) and/or a fraction thereof or a composition comprising same, in an effective amount sufficient to protect the tissue comprising epithelial cells from the adverse effects of the at least one pollutant, thereby protecting the tissue from the at least one pollutant. In a particular embodiment, the effective amount sufficient to protect the tissue comprising epithelial cells from adverse effects of at least one pollutant ranges from supplementing a solution or composition used for administering the extract with 0.1% to 20.0% weight per weight (w/w) polar solvent extract derived from an Inula plant as described herein. In a particular embodiment, the effective amount sufficient to protect the tissue comprising epithelial cells from adverse effects of at least one pollutant ranges from supplementing a solution or composition used for administering the extract with 0.2% to 10.0% w/w polar solvent extract derived from an Inula plant as described herein. In another particular embodiment, the effective amount sufficient to protect the tissue comprising epithelial cells from adverse effects of at least one pollutant ranges from 0.25% to 5.0% w/w supplemental polar solvent extract derived from an Inula plant as described herein. In an even more particular embodiment, the effective amount sufficient to protect the tissue comprising epithelial cells from adverse effects of at least one pollutant ranges from 0.5% to 2.0% w/w supplemental polar solvent extract derived from an Inula plant as described herein. In an even more particular embodiment, the therapeutically effective amount sufficient to at least partially restore tissue function ranges from 1.0% to 2.0% w/w supplemental polar solvent extract derived from an Inula plant as described herein. The method may be evaluated by measuring at least one of: skin wrinkles, skin texture, skin suppleness, skin evenness, skin permeability, skin homogeneity, and skin tone.

According to another aspect, a method for treating a tissue comprising epithelial cells, wherein the tissue is damaged due to exposure to at least one pollutant is presented, the method comprising administering to a subject in need thereof a polar solvent extract derived from an Inula plant (e.g., Inula helenium) and/or a fraction thereof or a composition comprising same, in a therapeutically effective amount sufficient to at least partially restore tissue function, thereby treating the tissue damaged by the at least one pollutant. In a particular embodiment, the effective amount sufficient to protect the tissue comprising epithelial cells from adverse effects of at least one pollutant ranges from supplementing a solution or composition used for administering the extract with 0.1% to 20.0% w/w polar solvent extract derived from an Inula plant as described herein. In a more particular embodiment, the therapeutically effective amount sufficient to at least partially restore tissue function ranges from supplementing a solution or composition used for administering the extract with 0.2% to 10.0% w/w polar solvent extract derived from an Inula plant as described herein. In another particular embodiment, the therapeutically effective amount sufficient to at least partially restore tissue function ranges from 0.25% to 5.0% w/w supplemental polar solvent extract derived from an Inula plant as described herein. In an even more particular embodiment, the therapeutically effective amount sufficient to at least partially restore tissue function ranges from 0.5% to 2.0% w/w supplemental polar solvent extract derived from an Inula plant as described herein. In an even more particular embodiment, the therapeutically effective amount sufficient to at least partially restore tissue function ranges from 1.0% to 2.0% w/w supplemental polar solvent extract derived from an Inula plant as described herein. The method may be evaluated by measuring at least one of: skin wrinkles, skin texture, skin suppleness, skin evenness, skin permeability, skin homogeneity, and skin tone.

According to another aspect, a method for repairing a tissue comprising epithelial cells, wherein the tissue is damaged due to exposure to at least one pollutant is presented, the method comprising administering to a subject in need thereof a polar solvent extract derived from an Inula plant (e.g., Inula helenium) and/or a fraction thereof or a composition comprising same, in an effective amount sufficient to at least partially restore tissue function, thereby repairing the tissue damaged by the at least one pollutant. In a particular embodiment, the effective amount sufficient to protect the tissue comprising epithelial cells from adverse effects of at least one pollutant ranges from supplementing a solution or composition used for administering the extract with 0.1% to 20.0% polar solvent extract derived from an Inula plant as described herein. In a more particular embodiment, the effective amount sufficient to at least partially restore tissue function ranges from supplementing a solution or composition used for administering the extract with 0.2% to 10.0% polar solvent extract derived from an Inula plant as described herein. In another particular embodiment, the effective amount sufficient to at least partially restore tissue function ranges from 0.25% to 5.0% supplemental polar solvent extract derived from an Inula plant as described herein. In an even more particular embodiment, the effective amount sufficient to at least partially restore tissue function ranges from 0.5% to 2.0% supplemental polar solvent extract derived from an Inula plant as described herein. In an even more particular embodiment, the effective amount sufficient to at least partially restore tissue function ranges from 1.0% to 2.0% supplemental polar solvent extract derived from an Inula plant as described herein. The method may be evaluated by measuring at least one of: skin wrinkles, skin texture, skin suppleness, skin evenness, skin permeability, skin homogeneity, and skin tone.

In particular embodiments, the extract derived from an Inula plant (e.g., Inula helenium) and/or a fraction thereof or a composition comprising same is administered to the subject prior to, during, and/or after exposure to the at least one pollutant.

In particular embodiments, the source of the at least one pollutant is at least one of combustion gas, industrial pollution, and smoke. In a more particular embodiment, the pollutant is a smoke-associated pollutant. In a still more particular embodiment, the source of the pollutant is tobacco smoke.

In another embodiment, the subject is expected to be exposed or is exposed or has been exposed to smoke. In a more particular embodiment, the smoke is a tobacco smoke. A subject exposed to smoke may be a tobacco smoker or may be exposed to tobacco smoke via secondhand exposure. A subject exposed to secondhand smoke may be referred to herein as a passive smoker.

In a particular embodiment, the subject is exposed to smoke sporadically. In another particular embodiment, the subject is exposed to smoke on a regular (e.g., daily) basis.

According to some embodiments, protecting the tissue from adverse effects of at least one pollutant results in increased target tissue viability, improved target tissue health, and reduced premature aging of target tissue. According to certain exemplary embodiments, the target tissue comprises epithelial cells. According to certain embodiments, protecting the target tissue comprises increasing the tolerance of tissue cells (e.g., epithelial cells therein) for the at least one pollutant.

According to certain embodiments, methods described herein are used to protect a target tissue. Exemplary target tissue includes: skin, nasal cavity, pharynx, larynx, trachea, and/or lung tissue.

According to certain exemplary embodiments, the epithelial tissue is the skin. According to these embodiments, the method comprises topically administering the polar solvent extract derived from an Inula plant (e.g., Inula helenium) and/or a fraction thereof or a composition comprising same to the skin of a subject in need thereof. According to further embodiments, protecting skin from adverse effects of at least one pollutant results in at least one of increased skin cell viability, improved skin appearance, and/or reduced premature skin aging. According to certain embodiments, improved skin appearance and/or reducing premature skin aging comprises reduction in the appearance of at least one of skin wrinkles, skin brown spots, and skin red spots. According to some embodiments, improving skin appearance and reducing premature skin aging comprises maintaining and/or improving the skin firmness and elasticity value, skin homogeneity, and skin impermeability as compared to a pre-determined threshold value.

According to additional exemplary embodiments, the epithelial tissue is tissue in at least one of the lung, trachea and nasal cavity. According to certain embodiments, the method comprises administering the polar solvent extract, and/or fraction thereof or a composition comprising same via inhalation. Additionally or alternatively, the method comprises administering the polar solvent extract, and/or fraction thereof or a composition comprising same by nozzle spray.

According to additional embodiments, the epithelial tissue forms part of at least one of pharynx and larynx. According to these embodiments, the polar solvent extract, and/or fraction thereof or composition comprising same is formulated for oral care.

According to some embodiments, the pollutant comes in contact with the tissue comprising epithelial cells in its isolated form. According to other embodiments, the pollutant is present in a medium coming in contact with the subject tissue.

According to certain embodiments, the pollutant and/or the medium comprising same comes in contact with the skin of the subject. According to other embodiments, the pollutant and/or the medium comprising same comes in contact with the oral cavity of the subject. According to additional embodiments, the pollutant and/or the medium comprising same comes in contact with the respiratory system of the subject.

According to certain embodiments, the pollutant is present in a medium selected from the group consisting of ambient air, water and soil.

According to some embodiments, the subject is human. According to certain exemplary embodiments, the human subject is a smoker.

According to certain embodiments, the compositions and methods described herein employ a fraction of the polar solvent extract. According to other embodiments, the compositions and methods described herein employ the complete polar solvent extract derived from Inula helenium.

According to a particular embodiment, the polar solvent extract and/or the fraction thereof is derived from the whole Inula helenium plant. According to a particular embodiment, the polar solvent extract and/or the fraction thereof is derived from the Inula helenium plant excluding the roots of the plant. According to some embodiments, the polar solvent extract and/or the fraction thereof is derived from any of the aerial parts of the Inula helenium plant. According to further embodiments, the aerial part is selected from the group consisting of a stem, a leaf, a seed, a flower, a fruit and any combination thereof. According to certain embodiments, the aerial part is a stem and/or a leaf. In a further embodiment, a polar solvent extract and/or the fraction thereof derived from the Inula helenium plant excluding the roots of the plant is combined with a polar solvent extract and/or the fraction thereof derived from Inula helenium plant roots. Such a combination of extracts may be present in a ratio of plant excluding the roots extract to root extract as follows: 90%:10%; 80%:20%; 70%:30%; 60%:40%; 50%:50%; 40%:60%; 30%:70%; 20%:80%; 10%:90%; and 5%:95%; and single digit increments thereof.

According to some embodiments, the polar solvent is water. According to other embodiments, the polar solvent comprises water and at least one additional polar solvent. According to certain embodiments, the additional polar solvent is selected from the group consisting of 1,2,3-trihydroxypropane (also known as glycerin or glycerol), ethanol, propylene glycol, butylene glycol, methanol, and acetone. In some embodiments, the polar solvent comprises water and glycerin. In other embodiments, the polar solvent comprises water and butylene glycol. In yet other embodiments, the polar solvent comprises propylene glycol. According to certain exemplary embodiments, the extract or the fraction thereof is a solution comprising water and glycerin, wherein the glycerin concentration ranges from 20% to 80% (w/w) relative to the total weight of the solution.

According to certain embodiments, the solution comprising the extract and/or fraction thereof described herein and glycerin is a clear, homogenous solution. According to some exemplary embodiments, the solution is stable for at least three months at a temperature of 40-50° C. without the formation of visible aggregates.

According to certain embodiments, the extract or the fraction thereof is essentially devoid of essential oils. According to some embodiments, the extract or the fraction thereof comprises up to 0.1%, 0.05%, 0.03%, 0.01%, 0.005%, or 0.001% w/w of essential oils. According to further embodiments, the extract or the fraction thereof is essentially devoid of oils or devoid of essential oils.

According to a further aspect, a composition or formulation is presented comprising a polar solvent extract derived from an Inula plant (e.g., Inula helenium) or a fraction thereof in an amount effective for protecting a tissue comprising epithelial cells from adverse effects of at least one pollutant or for treating a tissue comprising epithelial cells damaged by adverse effects of at least one pollutant, further comprising a cosmetically, and/or pharmaceutically acceptable and/or a dermatological diluent, excipient, or carrier.

In a particular embodiment, the Inula plant is Inula helenium.

According to some embodiments, the extract or the fraction thereof is derived from the whole Inula helenium plant or any of the aerial parts of the Inula helenium plant. In a particular embodiment thereof, the extract or the fraction thereof is derived from the Inula helenium plant without the root. According to further embodiments, the aerial part is selected from the group consisting of stem, leaf, seed, flower, fruit and any combination thereof. According to certain embodiments, the aerial part is a stem and/or a leaf.

According to certain embodiments, the extract or the fraction thereof is essentially devoid of essential oils. According to some embodiments, the extract fraction comprises up to 1%, 0.5%, 0.1%, 0.05%, 0.03%, 0.01%, 0.005%, or 0.001% w/w of essential oils. According to further embodiments, the extract or the fraction thereof is essentially devoid of oils or devoid of essential oils.

According to certain embodiments, the composition comprises the Inula extract and/or the fraction thereof at a concentration of 0.1% to 20% (w/w). According to other embodiments, the composition comprises the extract or the fraction thereof at a concentration of 0.1% to 10% (w/w). According to other embodiments, the composition comprises the extract or the fraction thereof at a concentration of 0.2% to 10% (w/w). According to other embodiments, the composition comprises the extract or the fraction thereof at a concentration of 0.2% to 5% (w/w). According to other embodiments, the composition comprises the extract or the fraction thereof at a concentration of 0.25% to 5% (w/w). According to other embodiments, the composition comprises the extract or the fraction thereof at a concentration of 1% to 5% (w/w). According to other embodiments, the composition comprises the extract or the fraction thereof at a concentration of 0.5% to 2% (w/w). According to other embodiments, the composition comprises the extract or the fraction thereof at a concentration of 1% to 2% (w/w). According to certain exemplary embodiments, the composition comprises the extract and/or the fraction thereof at a concentration of 1% (w/w) relative to the total weight of the composition.

According to some embodiments, the composition further comprises at least one additional plant extract. According to other embodiments, the composition further comprises at least one microalgae extract.

According to some embodiments, the composition further comprises an additional active agent comprising an anti-oxidant, a chelator, a cleansing agent, a skin protectant, a sunscreen, a skin lightening agent, additional an anti-wrinkling agent, an anti-inflammatory agent, anti-aging agent, or any combination thereof.

According to some embodiments, the composition is formulated in an aqueous solution, cream, lotion, water in oil emulsion, oil in water emulsion, microemulsion, nanoemulsion, gel, serum or milk.

According to certain embodiments, the composition is formulated for topical administration. Any topical formulation as is known in the art can be used according to the teachings of the present invention.

According to certain embodiments, the composition is a cosmetic or dermatological composition further comprising a cosmetically acceptable or dermatological carrier, diluent and/or excipient.

According to some embodiments, the cosmetically acceptable or dermatological carrier comprises a liposome, a micelle structure, a microcapsule, or any combination thereof.

According to some embodiments, the cosmetic composition further comprises a cosmetically acceptable or a dermatological additive. According to certain embodiments, the additive includes fats, emulsifiers, co-emulsifiers, hydrophilic gelling agents, lipophilic gelling agents, preservatives, solvents, fragrances, fillers, hydrophilic filters, lipophilic filters, dyestuffs, pigments, neutralizers, pH buffers, penetration-enhancing agents, emollients, polymers or any combination thereof.

According to other embodiments, the composition is formulated for administration via a spray nozzle.

According to some embodiments, the composition is an inhalation composition. According to certain embodiments, the inhalation composition is delivered to the respiratory tract by employing inhalation devices. According to certain embodiments, the inhalation device is an inhaler or a nebulizer. According to certain embodiments, the inhalation composition is administered by humidifier. According to other embodiments, the inhalation composition is administered through an air filtration system.

According to some embodiments, the composition is an oral care composition. According to certain embodiments, the oral care composition is a toothpaste, dental cream, gel, powder, mouth wash, breath freshener, gingival massage cream, gargling tablet, lozenge, or chewing gum. According to certain embodiments, the oral care composition comprises an additional agent comprising white colorants, whitening agents, enzymes, anti-plaque agents, anti-staining agents, anti-microbial agents, anti-caries agents, flavoring agents, coolants, or salivating agents. According to some embodiments, the oral care composition further comprises a reagent comprising abrasives, surfactants, chelating agents, fluoride sources, thickening agents, buffering agents, solvents, humectants, carriers, or bulking agents.

According to additional embodiments, each and every extract and/or fraction thereof and each and every composition of same described herein is for use in protecting a tissue comprising epithelial cell from adverse effects of a pollutant and/or in reducing, at least in part, the adverse effects of exposure to a pollutant. According to certain embodiments, the tissue is selected from the skin, nasal cavity, pharynx, larynx, trachea, and/or lung tissue. In an embodiment thereof, the composition comprises a polar solvent extract derived from Inula plant or a fraction thereof. Polar solvent extracts derived from Inula plants or a fraction thereof are as described herein.

In a further aspect, a polar solvent extract derived from an Inula plant and/or a fraction of the polar solvent extract or a composition comprising a polar solvent extract derived from an Inula plant and/or a fraction of the polar solvent extract is for use in protecting a tissue comprising epithelial cells from adverse effects of at least one pollutant and/or at least partially restoring tissue function of a tissue comprising epithelial cells damaged by exposure to adverse effects of at least one pollutant. In an embodiment thereof, the Inula plant is Inula helenium. In an embodiment thereof, the polar solvent extract and/or the fraction thereof is derived from the whole Inula helenium plant. According to a particular embodiment, the polar solvent extract and/or the fraction thereof is derived from the Inula helenium plant excluding the roots of the plant. According to some embodiments, the polar solvent extract and/or the fraction thereof is derived from any of the aerial parts of the Inula helenium plant. According to further embodiments, the aerial part is selected from the group consisting of a stem, a leaf, a seed, a flower, a fruit and any combination thereof. According to certain embodiments, the aerial part is a stem and/or a leaf. In a further embodiment, a polar solvent extract and/or the fraction thereof derived from the Inula helenium plant excluding the roots of the plant is combined with a polar solvent extract and/or the fraction thereof derived from Inula helenium plant roots. Such a combination of extracts may be present in a ratio of plant excluding the roots extract to root extract as follows: 90%:10%; 80%:20%; 70%:30%; 60%:40%; 50%:50%; 40%:60%; 30%:70%; 20%:80%; 10%:90%; and 5%:95%; and single digit increments thereof. According to some embodiments, the polar solvent is water. According to other embodiments, the polar solvent comprises water and at least one additional polar solvent. According to certain embodiments, the additional polar solvent is selected from the group consisting of 1,2,3-trihydroxypropane (also known as glycerin or glycerol), ethanol, propylene glycol, butylene glycol, methanol, and acetone. In some embodiments, the polar solvent comprises water and glycerin. In other embodiments, the polar solvent comprises water and butylene glycol. In yet other embodiments, the polar solvent comprises propylene glycol. According to certain exemplary embodiments, the extract or the fraction thereof is a solution comprising water and glycerin, wherein the glycerin concentration ranges from 20% to 80% (w/w) relative to the total weight of the solution. According to certain embodiments, the solution comprising the extract and/or fraction thereof described herein and glycerin is a clear, homogenous solution. According to some exemplary embodiments, the solution is stable for at least three months at a temperature of 40-50° C. without the formation of visible aggregates. According to certain embodiments, the extract or the fraction thereof is essentially devoid of essential oils. According to some embodiments, the extract or the fraction thereof comprises up to 0.1%, 0.05%, 0.03%, 0.01%, 0.005%, or 0.001% w/w of essential oils. According to further embodiments, the extract or the fraction thereof is essentially devoid of oils or devoid of essential oils. According to a further aspect, the use of a polar solvent extract derived from an Inula plant (e.g., Inula helenium) or a fraction thereof is in an amount effective for protecting a tissue comprising epithelial cells from adverse effects of at least one pollutant or for treating or at least partially restoring function to a tissue comprising epithelial cells damaged by adverse effects of at least one pollutant, and such use further comprises a cosmetically, and/or pharmaceutically acceptable and/or a dermatological diluent, excipient, or carrier. According to certain embodiments, the use comprises the Inula extract and/or the fraction thereof at a concentration of 0.1% to 20% (w/w). According to other embodiments, the use comprises the extract or the fraction thereof at a concentration of 0.1% to 10% (w/w). According to other embodiments, the use comprises the extract or the fraction thereof at a concentration of 0.2% to 10% (w/w). According to other embodiments, the use comprises the extract or the fraction thereof at a concentration of 0.2% to 5% (w/w). According to other embodiments, the use comprises the extract or the fraction thereof at a concentration of 0.25% to 5% (w/w). According to other embodiments, the use comprises the extract or the fraction thereof at a concentration of 1% to 5% (w/w). According to other embodiments, the use comprises the extract or the fraction thereof at a concentration of 0.5% to 2% (w/w). According to other embodiments, the use comprises the extract or the fraction thereof at a concentration of 1% to 2% (w/w). According to certain exemplary embodiments, the use comprises the extract and/or the fraction thereof at a concentration of 1% (w/w) relative to the total weight of the composition. According to some embodiments, the use further comprises at least one additional plant extract. According to other embodiments, the use further comprises at least one microalgae extract. According to some embodiments, the use further comprises an additional active agent comprising an anti-oxidant, a chelator, a cleansing agent, a skin protectant, a sunscreen, a skin lightening agent, additional an anti-wrinkling agent, an anti-inflammatory agent, anti-aging agent, or any combination thereof. According to some embodiments, the extract or the fraction thereof is formulated in an aqueous solution, cream, lotion, water in oil emulsion, oil in water emulsion, microemulsion, nanoemulsion, gel, serum or milk. According to certain embodiments, the extract or the fraction thereof is formulated for topical administration. Any topical formulation as is known in the art can be used according to the teachings of the present invention. According to certain embodiments, the extract or the fraction thereof is used in a cosmetic or dermatological composition further comprising a cosmetically acceptable or dermatological carrier, diluent and/or excipient. According to some embodiments, the cosmetically acceptable or dermatological carrier comprises a liposome, a micelle structure, a microcapsule, or any combination thereof. According to some embodiments, the cosmetic composition further comprises a cosmetically acceptable or a dermatological additive. According to certain embodiments, the additive includes fats, emulsifiers, co-emulsifiers, hydrophilic gelling agents, lipophilic gelling agents, preservatives, solvents, fragrances, fillers, hydrophilic filters, lipophilic filters, dyestuffs, pigments, neutralizers, pH buffers, penetration-enhancing agents, emollients, polymers or any combination thereof. According to other embodiments, the composition is formulated for use via a spray nozzle. According to some embodiments, the composition is for use as an inhalation composition. According to certain embodiments, the inhalation composition is for delivery to the respiratory tract by employing inhalation devices. According to certain embodiments, the inhalation device is an inhaler or a nebulizer. According to certain embodiments, the inhalation composition is for delivery by a humidifier. According to other embodiments, the inhalation composition is for delivery through an air filtration system. According to some embodiments, the composition is for use as an oral care composition. According to certain embodiments, the oral care composition is a toothpaste, dental cream, gel, powder, mouth wash, breath freshener, gingival massage cream, gargling tablet, lozenge, or chewing gum. According to certain embodiments, the oral care composition comprises an additional agent comprising white colorants, whitening agents, enzymes, anti-plaque agents, anti-staining agents, anti-microbial agents, anti-caries agents, flavoring agents, coolants, or salivating agents. According to some embodiments, use of the oral care composition further comprises a reagent comprising abrasives, surfactants, chelating agents, fluoride sources, thickening agents, buffering agents, solvents, humectants, carriers, or bulking agents.

In a further aspect, a polar solvent extract derived from an Inula plant and/or a fraction of the polar solvent extract for use in the preparation of a medicament for protecting a tissue comprising epithelial cells from adverse effects of at least one pollutant and/or for at least partially restoring tissue function of a tissue comprising epithelial cells damaged by exposure to adverse effects of at least one pollutant. In an embodiment thereof, the Inula plant is Inula helenium. In an embodiment thereof, the polar solvent extract and/or the fraction thereof is derived from the whole Inula helenium plant. According to a particular embodiment, the polar solvent extract and/or the fraction thereof is derived from the Inula helenium plant excluding the roots of the plant. According to some embodiments, the polar solvent extract and/or the fraction thereof is derived from any of the aerial parts of the Inula helenium plant. According to further embodiments, the aerial part is selected from the group consisting of a stem, a leaf, a seed, a flower, a fruit and any combination thereof. According to certain embodiments, the aerial part is a stem and/or a leaf. In a further embodiment, a polar solvent extract and/or the fraction thereof derived from the Inula helenium plant excluding the roots of the plant is combined with a polar solvent extract and/or the fraction thereof derived from Inula helenium plant roots. Such a combination of extracts may be present in a ratio of plant excluding the roots extract to root extract as follows: 90%:10%; 80%:20%; 70%:30%; 60%:40%; 50%:50%; 40%:60%; 30%:70%; 20%:80%; 10%:90%; and 5%:95%; and single digit increments thereof. According to some embodiments, the polar solvent is water. According to other embodiments, the polar solvent comprises water and at least one additional polar solvent. According to certain embodiments, the additional polar solvent is selected from the group consisting of 1,2,3-trihydroxypropane (also known as glycerin or glycerol), ethanol, propylene glycol, butylene glycol, methanol, and acetone. In some embodiments, the polar solvent comprises water and glycerin. In other embodiments, the polar solvent comprises water and butylene glycol. In yet other embodiments, the polar solvent comprises propylene glycol. According to certain exemplary embodiments, the extract or the fraction thereof is a solution comprising water and glycerin, wherein the glycerin concentration ranges from 20% to 80% (w/w) relative to the total weight of the solution. According to certain embodiments, the solution comprising the extract and/or fraction thereof described herein and glycerin is a clear, homogenous solution. According to some exemplary embodiments, the solution is stable for at least three months at a temperature of 40-50° C. without the formation of visible aggregates. According to certain embodiments, the extract or the fraction thereof is essentially devoid of essential oils. According to some embodiments, the extract or the fraction thereof comprises up to 0.1%, 0.05%, 0.03%, 0.01%, 0.005%, or 0.001% w/w of essential oils. According to further embodiments, the extract or the fraction thereof is essentially devoid of oils or devoid of essential oils. According to a further aspect, the use of a polar solvent extract derived from an Inula plant (e.g., Inula helenium) or a fraction thereof is in an amount effective for protecting a tissue comprising epithelial cells from adverse effects of at least one pollutant or for treating or at least partially restoring function to a tissue comprising epithelial cells damaged by adverse effects of at least one pollutant, and such a medicament further comprises a cosmetically, and/or pharmaceutically acceptable and/or a dermatological diluent, excipient, or carrier. According to certain embodiments, the medicament comprises the Inula extract and/or the fraction thereof at a concentration of 0.1% to 20% (w/w). According to other embodiments, the medicament comprises the extract or the fraction thereof at a concentration of 0.1% to 10% (w/w). According to other embodiments, the medicament comprises the extract or the fraction thereof at a concentration of 0.2% to 10% (w/w). According to other embodiments, the medicament comprises the extract or the fraction thereof at a concentration of 0.2% to 5% (w/w). According to other embodiments, the medicament comprises the extract or the fraction thereof at a concentration of 0.25% to 5% (w/w). According to other embodiments, the medicament comprises the extract or the fraction thereof at a concentration of 1% to 5% (w/w). According to other embodiments, the medicament comprises the extract or the fraction thereof at a concentration of 0.5% to 2% (w/w). According to other embodiments, the medicament comprises the extract or the fraction thereof at a concentration of 1% to 2% (w/w). According to certain exemplary embodiments, the medicament comprises the extract and/or the fraction thereof at a concentration of 1% (w/w) relative to the total weight of the composition. According to some embodiments, the medicament further comprises at least one additional plant extract. According to other embodiments, the medicament further comprises at least one microalgae extract. According to some embodiments, the medicament further comprises an additional active agent comprising an anti-oxidant, a chelator, a cleansing agent, a skin protectant, a sunscreen, a skin lightening agent, additional an anti-wrinkling agent, an anti-inflammatory agent, anti-aging agent, or any combination thereof. According to some embodiments, the extract or the fraction thereof is formulated in a medicament comprising an aqueous solution, cream, lotion, water in oil emulsion, oil in water emulsion, microemulsion, nanoemulsion, gel, serum or milk. According to certain embodiments, the extract or the fraction thereof is formulated in a medicament for topical administration. Any topical formulation as is known in the art can be used according to the teachings of the present invention. According to certain embodiments, the extract or the fraction thereof is used in the preparation of a medicament, such as a cosmetic or dermatological composition further comprising a cosmetically acceptable or dermatological carrier, diluent and/or excipient. According to some embodiments, the cosmetically acceptable or dermatological carrier comprises a liposome, a micelle structure, a microcapsule, or any combination thereof. According to some embodiments, the cosmetic composition further comprises a cosmetically acceptable or a dermatological additive. According to certain embodiments, the additive includes fats, emulsifiers, co-emulsifiers, hydrophilic gelling agents, lipophilic gelling agents, preservatives, solvents, fragrances, fillers, hydrophilic filters, lipophilic filters, dyestuffs, pigments, neutralizers, pH buffers, penetration-enhancing agents, emollients, polymers or any combination thereof. According to other embodiments, the medicament is formulated for use via a spray nozzle. According to some embodiments, the medicament is for use as an inhalation composition. According to certain embodiments, the inhalation composition is for delivery to the respiratory tract by employing inhalation devices. According to certain embodiments, the inhalation device is an inhaler or a nebulizer. According to certain embodiments, the inhalation composition is for delivery by a humidifier. According to other embodiments, the inhalation composition is for delivery through an air filtration system. According to some embodiments, the medicament is for use as an oral care composition. According to certain embodiments, the oral care composition is a toothpaste, dental cream, gel, powder, mouth wash, breath freshener, gingival massage cream, gargling tablet, lozenge, or chewing gum. According to certain embodiments, the oral care composition comprises an additional agent comprising white colorants, whitening agents, enzymes, anti-plaque agents, anti-staining agents, anti-microbial agents, anti-caries agents, flavoring agents, coolants, or salivating agents. According to some embodiments, use of the oral care composition further comprises a reagent comprising abrasives, surfactants, chelating agents, fluoride sources, thickening agents, buffering agents, solvents, humectants, carriers, or bulking agents.

Other objects, features and advantages of the present invention will become clear from the following description and examples.

### BRIEF DESCRIPTION OF THE FIGURES

FIG. 1 presents a histogram depicting the detoxification effects of Inula helenium (I. helenium) extract in an aqueous cell culture model of Dunaliella salina (D. salina). D. salina cultures were exposed to various toxins, including methanol at 60,000 ppm, gasoline (95-octane) at 500 ppm, and formaldehyde at 25 ppm, for 48 hours. The protective effect of the I. helenium extract was tested at concentrations of 1% and 2%.

FIG. 2 depicts the protective effects of I. helenium extract in an aqueous cell culture model of D. salina, wherein D. salina cells are exposed to the toxin gasoline. The effect of the extract was tested at concentrations of 1% and 2%. I. helenium extract confers protection against the toxic effects of gasoline as reflected by increased cell viability in the presence of the extract relative to that of a negative control (0% extract).

FIG. 3 presents microscope images illustrating the protective effects of I. helenium extract in an aqueous cell culture model of D. salina, wherein D. salina cells are exposed to the toxin gasoline. The effect of the extract was tested at concentrations of 1% and 2%. I. helenium extract confers protection against the toxic effects of gasoline as reflected by increased cell viability in the presence of the extract relative to that of a negative control (0% extract). When viewed microscopically, dead cells are transparent, whereas viable cells are opaque and have a greenish tint. More live cells are observed in the presence of increasing amounts of extract. Images presented in this figure are representative. Viability of the algae has also been determined quantitatively by counting live algal cells (10 microscope fields per sample). The difference in viability in the presence of I. helenium extract relative to negative control is statistically significant.

FIG. 4A-4D presents a histogram depicting the effects of I. helenium extract on skin color determined using a chromameter.

FIG. 5A-5D presents a histogram depicting the effects of I. helenium extract on skin wrinkles determined using Primos.

FIG. 6 presents a histogram depicting the effects of I. helenium extract on skin wrinkles determined by clinical grading.

FIG. 7A-7B presents a histogram depicting the effects of I. helenium extract on skin texture and suppleness as determined by clinical grading.

FIG. 8A-8C presents a histogram depicting the effects of I. helenium extract on skin wrinkles, evenness, and brown spots determined using Visia.

FIG. 9 presents a histogram depicting the effects of I. helenium extract on the ability of skin to act as a barrier determined by evaluating transepidermal water loss (TEWL).

FIG. 10 presents a histogram depicting the effects of I. helenium extract on skin texture determined via Visioscan.

FIG. 11A-11B presents a histogram of results derived from self-evaluating questionnaires completed by clinical subjects relating to the effects of I. helenium extract on skin wrinkles and tone.

FIG. 12 presents a histogram depicting the effects of I. helenium extract on skin elasticity (R2) variation periorbital.

FIG. 13 presents a histogram depicting the effects of I. helenium extract on skin elasticity variation around the corner of the mouth.

FIG. 14 presents a histogram of results depicting the inhibitory effect of I. helenium extract on high energy visible (HEV) light-induced reactive oxygen species (ROS) production in human keratinocytes (HaCaT).

FIG. 15 presents a histogram depicting the metal chelating activity of extracts made from Inula species indicated therein. I. helenium extract demonstrates significantly more robust metal-chelating activity relative to other Inula species extracts.

### DETAILED DESCRIPTION OF THE INVENTION

The genus Inula is a diverse genus that includes about 90 species of flowering plants. One of these species, Inula helenium (I. helenium) is an herb, widely distributed throughout Europe where it was cultivated for centuries as a medicinal plant, being a common remedy for various illnesses in the Middle Ages. The plant is also found in temperate Asia as far as southern Siberia and northwestern India and has been cultivated in North America as well. I. helenium is a striking, handsome plant that is in bloom during the summer. The flowers are bright yellow and presented in very large, terminal heads (3 to 4 inches in diameter) on long stalks. I. helenium flowers resemble a double sunflower. The broad bracts of the leafy involucre under the head are velvety. I. helenium plant is a perennial plant.

Its rootstock is large and succulent, having a spindle-shaped and branching morphology, brown color, and aromatic fragrance. Its large, fleshy roots have been used as medicines and condiments. Most pharmaceutical preparations from Inula are made of the two to three year old roots which are dug in autumn. The root has oil-glands and is distinguished by its yellow, radiate wood, distinctive odor, and taste.

As described herein, extracts made from I. helenium plants, excluding the roots, surprisingly exhibit the ability to counteract pollution and its effects on organs and tissues comprising epithelial cells. As such, I. helenium plant extracts described herein and compositions/formulations thereof exhibit properties characteristic of anti-pollutant agents. Results presented herein demonstrate that I. helenium plant extracts can act as detoxification agents in polluted aquatic culture assays, as anti-chelation agents in in vitro assays, and as therapeutically effective agents in clinical trials demonstrating improved skin condition in smokers treated with I. helenium plant extract compositions. These results further suggest that I. helenium plant extracts promote maintenance of a healthy state of organs and tissues comprising epithelial cells by maintaining and restoring a local environment wherein epithelial cells can optimally access nutrients from surrounding tissue and beneficial microorganisms can thrive due to a reduction in exogenous pollutants (e.g., toxins) that impair normal metabolic processes. Accordingly, I. helenium plant extracts described herein and compositions thereof inhibit the adverse effects of a variety of pollutants and thus, may be referred to herein as pollutant inhibitors. As such, I. helenium plant extracts described herein and compositions thereof satisfy a long sought need to combat the effects of pollutants and generally promote a healthy skin condition conferring anti-ageing benefits, including improved epithelial cell health, increased skin hydration, and a reduction in wrinkles and skin pigmentation.

Results presented herein, therefore, demonstrate that I. helenium plant extracts described herein and compositions thereof meet the need to have improved compositions and methods effective for protecting and preventing adverse effects of environmental hazards, particularly by pollutants, on organs and tissues comprising epithelial tissues in general and skin in particular.

Skin is the largest and the most exposed organ of the body comprising epithelial tissues. Skin has numerous functions, the primary function being a protective barrier against harmful physical forces, chemicals, pollutants, pathogens and other undesired agents. The skin consists of three main layers: the epidermis, the outermost layer of skin, which provides a waterproof barrier and creates our skin tone; the underlying dermis, which comprises tough connective tissue, hair follicles, and sweat glands; and the deeper subcutaneous tissue (hypodermis), which is made of fat and connective tissue. The epidermis is further subdivided into several layers: the stratum corneum, the stratum granulosum, the stratum spinosum, and the stratum basale.

The skin comprises stratified squamous, keratinized epithelial cells and viable keratinocytes among other cell types. Tissues lining the inside of the mouth, the esophagus and part of the rectum also comprise non-keratinized stratified squamous epithelium. Other surfaces that separate body cavities from the outside environment are padded by simple squamous, columnar, or pseudo-stratified epithelial cells. Other epithelial cells line the inside of the lungs, the gastrointestinal tract, the reproductive and urinary tracts, and make up the exocrine and endocrine glands. The outer surface of the cornea is covered with fast-growing, easily regenerated epithelial cells.

In these organs and tissues, the epithelium serves as part of the protective barrier against external challenges, including, for example, harmful physical forces, chemicals, pollutants, and pathogens. The epithelium, therefore, provides a first line of defense in protecting the body from these challenges.

Pollution born toxins have detrimental effects on the health of cells and tissues and organs comprising epithelial cells. Pollution may be caused by many diverse factors, including, for example, indoor pollution resulting from cigarette smoke, cleaning products and dust, and outdoor pollution due to engine smoke, chemical substances, industrial waste, and carbon monoxide.

Toxic buildup is known to impair an organ's normal physiologic functions. The constant, sometimes daily exposure to environmental toxins leads to accumulated damage in the cells of all exposed organs. Common cellular mechanisms by which most pollutants exert their adverse effects include their ability to act directly as pro-oxidants of lipids and proteins; form DNA adducts, DNA breakage, or mutations; and/or to act as free radical generators, promoting oxidative stress and the induction of inflammatory responses.

Pollutants mainly reach mammalian tissues via dermal contact, inhalation, and ingestion of contaminated products. Air and soil pollution contributes, to a great extent, to the contamination of food and water, such that ingestion also provides a means for pollutant intake. Pollutants deposited in the gastrointestinal and respiratory tracts may be absorbed by the epithelial cells such that toxic substances appear in the general circulation and accumulate in different tissues.

The link between exposure to a certain substances and epithelium damage is universally accepted. For example, formaldehyde is now recognized worldwide as both a carcinogen and a skin sensitizer. The pollutant chemicals found in tobacco smoke are also associated with detrimental effects on epithelial cells. Cigarette smoke contains over 4,700 chemical compounds of which about 60 are carcinogenic. People who smoke on a regular basis are prone to damage of epithelial cell comprising organs and tissues, particularly the lung, oral cavity, lips, and skin. Aging processes of the skin of smokers are also accelerated.

All types of pollution, at high concentration, can affect mammalian airways and gastrointestinal tract. Nevertheless, similar effects are also observed with long-term exposure to lower pollutant concentrations. Symptoms such as nose and throat irritation, followed by bronchoconstriction and dyspnea, especially in asthmatic individuals, are usually experienced after exposure to increased levels of sulphur dioxide, nitrogen oxides, and certain heavy metals such as arsenic, nickel or vanadium. Air pollutants such as nitrogen oxides increase the susceptibility to respiratory infections. Chronic exposure to ozone and certain heavy metals reduces lung function and is linked to asthma, emphysema, and even lung cancer. Emphysema-like lesions have also been observed in mice exposed to nitrogen dioxide

The epithelium possesses a limited active toxin defense e.g., physical removal of exogenous toxins by several routes. Exemplary routes include the natural expulsion of contaminated cells as part of the skin's organic turn-over and dissolution of toxins from interstitial spaces and transport back to the skin surface via sweat production and secretion. However, the natural defense mechanisms against toxins do not provide full protection.

Toxins that have not been physically removed may be metabolized within the epithelium. In recent years it has become clear that human epithelium cells express various Cytochrome P450 (CYP) enzymes, including those responsible specifically for metabolism of exogenous toxins within various types of epithelial cells. The resulting metabolites also potentially damage the epithelium and body. Defense against skin toxins depends on several factors, including behavioral routines (i.e., avoidance of exposure, diet) and use of traditional medicine, therapeutic and/or cosmetic products.

Traditional medicine has long appreciated the negative effect of toxin deposit on health and well being and practices detoxification of the body as standard treatment for many diseases and disorders. Similar practices, with some variations, are very commonly self-implemented by the public. These detoxification processes focus on reducing the exposure to the toxin and increased removal of the toxin from the body.

The present invention provides an extract derived from I. helenium and/or fractions thereof and/or compositions comprising same for preventing and/or treating organs and tissues comprising epithelial cells that are at risk for or have been damaged by pollutants.

The present invention is based in part on the unexpected discovery that a polar solvent extract of I. helenium plants excluding roots protects cultured Dunaliella salina (D. salina) cells from water-borne toxins in an in vitro model of human epithelial cell sensitivity to such toxins. Moreover, when applied to skin exposed to smoke in vivo (by topical application to the skin of heavy smoker subjects) the extract caused a reduction in wrinkle count, along with improvements in skin tone, texture, suppleness, and impermeability.

Without wishing to be bound by any particular theory or mechanism of action the high efficacy of the extracts and formulations/compositions thereof described herein to protect and/or increase tolerance of epithelial cells to damage caused by pollutants, particularly smoke-related pollutants, may be attributed to its ability to affect gene expression. The extracts and formulations/compositions thereof described herein may reduce the expression of genes encoding proteins that promote processes relating to inflammation, oxidation, and cell death. In a non-mutually exclusive embodiment, the extracts and formulations/compositions thereof described herein may increase the expression of at least one gene encoding a protein that promotes toxin metabolism. The active contribution of the extract to the cell protective mechanism may be attributed to particular components of the I. helenium plant that are soluble in polar solvents and/or to the extracting methods that preserve the plant-component protective qualities.

The extracts and compositions described herein are useful when implemented in methods for preventing and treating damage to epithelial tissues caused by various pollutants. The extracts and compositions described herein are also envisioned for use in the preventing and/or treating epithelial tissues (e.g., the skin) at risk for exposure to pollutants or damaged by exposure to pollutants. Extracts and compositions described herein are further useful in preventing and/or treating air pollution damage to epithelial tissues caused by fire smoke, engine combustion gas, and industrial pollution. Cigarette smoke is, for example, a source of air pollution. Numerous studies have shown the connection between toxins found in cigarette smoke and damage to the epithelial cells of lungs and skin. Cigarette smoke contains over 4,700 chemical compounds of which about 60 are widely recognized as carcinogenic. Cigarette smoke can thus serve as a broad model of airborne pollution.

Various treatment regimens are known in the art and encompassed herein. Compositions comprising extracts described herein may, for example, be administered to tissues comprising epithelial cells once or twice per day. Treatment regimen may be maintained indefinitely and perpetuated as long as the subject is exposed to pollutants. In particular embodiments, a subject may be treated for a one-ten week period of time. Treatment may also be intermittent. Treatment regimen may be evaluated and altered based on the subject's or a clinician's assessment of responsiveness to same

### Definitions

The terms “polar solvent extract” and “polar solvent extract derived from an Inula plant” and “an extract derived from an Inula plant wherein the extract is soluble in a polar solvent” are used herein interchangeably and refer to an extract obtained by extracting any part of at least one Inula species plant with a polar solvent. In a particular embodiment, the extract is made from at least one entire or whole Inula plant. In another particular embodiment, the extract is made from any part of at least one Inula helenium plant. In a more particular embodiment, the extract is made from the aerial parts only (everything but the roots) of at least one Inula plant. In a still more particular embodiment, the extract is made from only leaves of at least one Inula plant. The term “fraction thereof” refers to at least one fraction of the extract. According to certain exemplary embodiments, the fraction is obtained by filtering source crude extract.

As used herein, a fraction of a polar solvent extract derived from the Inula helenium plant or a portion of the plant may range anywhere from 1% to 99% of the total weight of the polar solvent extract. Accordingly, a fraction of the polar solvent extract may be 1%, 5%, 10%; 20%; 30%; 40%; 50%; 60%; 70%; 80%; 90%; or 95% of the total polar solvent extract (weight/weight); and single digit increments ranging from 1% to 99% of the total polar solvent extract. Each of these percent amounts may be obtained by filtering source crude extract.

The term “adverse effect” with reference to the at least one pollutant refers to any harm caused to a tissue comprising epithelial cells, particularly a tissue lining both the outer surface (skin) and the inside cavities and lumen of the body. The adverse effect may include, without limitation, loss of vitality, a metabolic disorder, premature aging and/or cell death. The term “adverse effect on skin” as used herein refers to any harm caused to the skin by pollutants as described herein, for example, premature skin aging, loss of vitality, and deterioration in the appearance of the skin, particularly as evidenced by the formation or deepening of wrinkles.

The term “skin damage” as used herein refers to harm to any of the skin layers as a result of exposure to pollutants as described herein. The term “pollutant” as used herein refers to a compound introduced into the environment that has undesired effects, or that adversely affects the characteristic and function of the medium to which it has been introduced. As used herein the term “pollutant” mainly refers to a xeno chemical compound that is harmful and/or poisonous to an organism (e.g., a human). According to certain embodiments, the pollutant is a smoke-related substance, particularly a smoke-related substance that damages epithelial cells. According to certain exemplary embodiments, the smoke is tobacco smoke.

The term “smoker” is used herein in its broadest scope and refers to a subject who smokes cigarettes, pipes, and/any other commonly used smoking material.

As used herein, the term “secondary or passive smoker” refers to a subject exposed to tobacco smoke.

The terms “epithelium”, “epithelial tissues”, and “epithelial layer” are used herein interchangeably and are meant to refer to the membranous tissue composed of one or more layers of epithelial cells separated by very little intercellular substance and forming the covering of most internal and external surfaces of the body and its organs. The term explicitly includes viable keratinocytes. The term “tissue comprising epithelial cells” refers to a tissue comprising epithelial cells as part of its structure. The epithelial cells according to the invention are found inter alia in the skin, oral and nasal cavities, pharynx, larynx, esophagus and lungs.

As used herein the term “essential oil” refers to volatile oil derived from leaves, stems, flowers, twigs, or roots, or a whole Inula helenium plant. Essential oils are hydrophobic oils derived from plant matter and substantially devoid of lipids or lipid oils.

The terms “essentially devoid of oil” and “essentially devoid of essential oil” are used herein in reference to the extract of the present invention to define an extract or a fraction thereof comprising oil or essential oil in an amount below the detectable level by a method common in the art. According to certain embodiments, the polar solvent extract of the invention or a fraction thereof comprises less than 1% oil or less than 1% essential oil. According to certain additional embodiments, the polar solvent extract of the invention or the fraction thereof comprises less than 0.1% oil or less than 0.1% essential oil.

As used herein, the term “and/or” includes any and all combinations of one or more of the associated listed items. Each possibility represents a separate embodiment of the invention.

As used herein, the percent or range thereof indicated with respect to the amount of polar solvent extract derived from an Inula plant in an extract, formulation, composition, or the like refers to the weight of polar solvent extract relative to the total weight of the extract, formulation, composition, or the like. Accordingly, such a percent or range thereof is expressed herein as weight/weight (w/w).

According to one aspect, the present invention provides a method for protecting a tissue comprising epithelial cells from the adverse effect of at least one pollutant, comprising administering to a subject in need thereof a polar solvent extract derived from an Inula plant or a fraction thereof, or a composition comprising same, thereby protecting the tissue from the at least one pollutant. Also encompassed herein is a polar solvent extract derived from an Inula plant or a fraction thereof, or a composition comprising same for use in treating and/or preventing damage from at least one pollutant to a tissue comprising epithelial cells.

According to certain embodiments, the subject is a mammal. According to certain exemplary embodiments, the subject is a human.

Inula helenium belongs to the Inula genus, which comprises about 90 different species. The fleshy root of the plant has been used as a medicine and a condiment. Unexpectedly, results presented herein reveal that a polar solvent extract of Inula helenium substantially devoid of essential oils, is useful for protecting tissue and/or organs comprising epithelial cells from pollutants, and/or for treating and/or preventing pollution-related damage to tissue and/or organs comprising epithelial cells, such as the skin.

Extracts described herein may be obtained from I. helenium plants grown in their natural habitat or, more particularly, from plants grown under designated agricultural conditions.

The extract may be obtained from any part of the plant or a whole plant. According to a particular embodiment, the extract is derived from at least one whole I. helenium plant. In another embodiment, the extract is derived from at least one I. helenium plant, excluding the plant roots. In another embodiment, the extract is derived from any of the aerial parts of the I. helenium plant. According to further embodiments, the aerial part comprises stems, leaves, seeds, fruit, or any combination thereof. According to certain embodiments, the aerial part is a stem or a leaf.

Various polar solvents known in the art can be used to obtain an extract described herein. According to certain exemplary embodiments, the polar solvent comprises water and at least one additional polar solvent selected from the group consisting of 1,2,3-trihydroxypropane (also known as glycerin or glycerol), ethanol, propylene glycol, butylene glycol, methanol, acetone, N,N-Dimethylformamide (DMF), acetonitrile (MeCN), dimethyl sulfoxide (DMSO), ammonia, t-Butanol, n-Propanol, acetic acid, isopropanol, and formic acid. Each possibility represents a separate embodiment of the present invention. Such polar solvents are known to persons of ordinary skill in the art, as are protocols for using same to obtain extracts such as those described herein.

According to certain exemplary embodiments, the pollutant-protecting components are extracted from a whole Inula species (e.g., I. helenium) plant using water as the polar solvent, with glycerin (1,2,3-trihydroxypropane) added to the aqueous medium after obtaining the desired extract fraction. According to these embodiments, an extract solution comprising at least one fraction of water extract of an Inula species (e.g., I. helenium) plant and glycerin is generated. According to some embodiments, the ratio of the at least one fraction of water extract of an Inula species (e.g., I. helenium) plant to glycerin is from 1:3 to 3:1. According to certain currently exemplary embodiments, the ratio is 1:1. This extract solution is clear, translucent, homogenous and highly stable for at least three months even when kept under elevated temperature of 40-50° C. without the formation of visible aggregates.

According to some embodiments, a tissue comprising epithelial cells is exposed to a pollutant in its isolated form. According to other embodiments, the pollutant is present in a medium coming in contact with the subject tissue.

As used herein, the term “pollution” refers to a medium comprising at least one pollutant.

The pollutant may be categorized to one or more groups selected from volatile organic compounds (VOCs), polyaromatic hydrocarbon (PHAs), oxides and heavy metals.

In some embodiments, the pollution is air pollution. In some embodiments, the air pollution is smoke. In other embodiments, the air pollution is tobacco smoke.

Many of the compounds found in cigarette smoke are also found in air-polluted cities and industrial areas. Hence, the composition of cigarette smoke is a representative example of the composition of air pollution.

According to certain exemplary embodiments, the pollutant is at least one of the pollutants presented in Table 1.

TABLE 1
Primary toxic and carcinogenic components of cigarette smoke
including vapor-phase and particulate phase components

| | | | | | Co-carcinogenic/ |
|---|---|---|---|---|---|
| Agent | Category | Toxic | Ciliotoxic | Carcinogenic | Promoter |
| Carbon Monoxide | Oxide | x | | | |
| Nitrogen Oxides (NOₓ) | Oxide | x | | | |
| Hydrogen Cyanide | VOC | x | x | | |
| Formaldehyde | VOC | | x | x | |
| Acrolein | VOC | | x | | |
| Acetaldehyde | VOC | | x | | |
| Ammonia | VOC | x | | | |
| Hydrazine | Leads to formation | | | x | |
| | of reactive oxygen | | | | |
| | species | | | | |
| Vinyl Chloride | VOC | | | x | |
| Urethane | VOC | | | x | |
| 2-Nitropropane | VOC | | | x | |
| Quinoline | PAH | | | x | |
| Benzo[a]pyrene | PAH | | | x | x |
| Dibenz[a,h]anthracene | PAH | | | x | x |
| Benzo[b]fluoranthene | PAH | | | x | x |
| Benzo[j]fluoranthene | PAH | | | x | x |
| Dibenzo[a,h]pyrene | PAH | | | x | x |
| Dibenzo[a,i]pyrene | PAH | | | x | x |
| Dibenz[a,j]acridine | PAH | | | x | x |
| Indeno[1,2,3-cd]pyrene | PAH | | | x | x |
| Benzo[c]phenanthrene | PAH | | | x | x |
| Benz[a]anthracene | PAH | | | x | x |
| Benzo[e]pyrene | PAH | | | x | x |
| Chrysene | PAH | | | x | x |
| Methylchrysene | PAH | | | x | x |
| Mehtylfluoranthene | PAH | | | x | x |
| Dibenz[a,c]anthracene | PAH | | | x | x |
| Dibenz[a,h]acridine | PAH | | | x | x |
| Dibenzo[c,g]carbazole | PAH | | | x | x |
| Mehtylnaphtalenes | PAH | | | | x |
| 1-Methylindoles | VOC | | | | x |
| Dichlorostilbene | PAH | | | | x |
| Catechol | PAH | | | | x |
| 3-Methycatechol | PAH | | | | x |
| 4-Methycatechol | PAH | | | | x |
| 4-Ethycatechol | PAH | | | | x |
| 4-n-Propylcatechol | PAH | | | | x |
| Nitrosodimethylamine | VOC | | | x | |
| Nitrosoethymethylamine | VOC | | | x | |
| Nitrosodiethylamine | VOC | | | x | |
| Nitrosodi-n-propylamine | VOC | | | x | |
| Nitrosodi-n-butylamine | VOC | | | x | |
| Nitrosopyrrolidine | VOC | | | x | |
| Nitrosopiperidine | VOC | | | x | |
| Nitrosomorpholine | VOC | | | x | |
| N′-Nitrosonomicotine | VOC | | | x | |
| 4-(methylnitrosamino)- | VOC | | | x | |
| 1-(3-pyridyl)-1-butanone | | | | | |
| N′-Nitrosoanabasine | VOC | | | x | |
| N′-Nitrosoanatabine | VOC | | | x | |
| Aromatic Amines | PAH | | | x | |
| Aromatic Nitrohydrocarbons | PAH | | | x | |
| Nickel | Heavy metal | | | x | |
| Arsenic | Heavy metal | | | x | |
| Cadmium | Heavy metal | | | x | |

Results presented herein reveal that polar solvent extracts of I. helenium, for example, are highly effective in protecting epithelial cells from adverse effects and/or damage due to exposure to pollutants, particularly smoke-associated pollutants. The extracts and compositions comprising same are thus useful in protecting subjects, particularly a human subject likely to be exposed or exposed to pollution, particularly smoke related pollution.

According to certain embodiments, the human subject is exposed to tobacco smoke. According to certain exemplary embodiments, the human subject is a heavy smoker.

The dosage of the extract or the fraction thereof and the route of administration depend on the target tissue to be protected, the degree of exposure to pollutant, and on characteristics of the subject, including gender, age and general health conditions, as is known to the skilled in the art.

According to certain exemplary embodiments, the extract, fraction thereof, or a composition comprising same is administered topically to a tissue selected from the group consisting of skin tissue, tracheal tissue, lung tissue and tissues within the oral cavity.

According to certain embodiments, the extract, fraction thereof, or a composition comprising same is administered topically to the skin. As used herein, the term “topical administration to skin” includes administration onto the skin surface as well as into the skin layers. According to certain embodiments, administration is performed transdermally and/or subcutaneously.

The polar solvent extract of I. helenium, for example, or a fraction thereof can be administered to a subject in need thereof per se, or in a composition comprising same.

In a further aspect, a composition is provided comprising a polar solvent extract derived from an Inula plant or a fraction thereof in an amount effective for protecting a tissue comprising epithelial cells from the adverse effects of at least one pollutant, further comprising a cosmetically, pharmaceutically or dermatologically acceptable diluent, excipient or carrier.

As described hereinabove, the extract described herein or a fraction thereof is intended for use in protecting epithelial cells within at least one of the skin, trachea, lung and the oral cavity. Any composition known in the art to be suitable for applying an active ingredient to skin, trachea, lung and oral cavity can be used according to the teachings of the invention, as long as the protecting activity of the extract of the invention against adverse effects of pollution to these tissues is preserved.

According to some embodiments, the composition is a cosmetic composition, an oral care composition, or an inhalation composition.

The term “cosmetic composition” refers to a composition suitable for topical application, particularly topical application onto skin in mammals. According to certain embodiments, the composition has a cosmetically beneficial effect upon the skin as described herein.

Table 2 presents an exemplary composition comprising Inula heleniwn extract for cosmetic use.

| INCI/chemical name | Tradename | % in formula |
|---|---|---|
| Water | Water | 84.35 |
| Butylene glycol | 1,3-butanediol | 4.00 |
| Dipropylene glycol | Dipropylene glycol | 1.00 |
| Hexylene glycol | Hexylene glycol | 1.00 |
| Polysorbate 20 | Tween 20 | 1.00 |
| Hydrogenated polydecene | Nexbase | 1.50 |
| Cyclomethicone | SF0005Z | 4.00 |
| Inula Helenium extract | Inula Helenium Extract | 1.00 |
| Glycerol | Glycerin | 0.00 |
| Carbomer | Carbopol 940 | 0.80 |
| Triethanolamine | Triethanolamine | 0.70 |
| Phenoxyethanol | Phenoxyethanol | 0.40 |
| Methyl paraben | Nipagin M | 0.15 |
| EDTA | EDTA | 0.10 |

| | |
|---|---|
| TOTAL | 100.00 |

Process for Making Cosmetic Composition on a Laboratory Scale:
1. Preservatives/polyols premix: the following are combined with stirring at 45° C. until complete dissolution: Butylene glycol; Hexylene glycol; Dipropylene glycol; Phenoxyethanol; Methyl paraben.
2. Organic phase: the following are combined with stirring at 45° C. until complete mixing: Cyclomethicone; Hydrogenated Polydecene.
3. Main mix:
   a. The Carbomer is dispersed into the Water (84.35% formula weight) at 45° C., slowly and with vigorous agitation. Agitation is continued until the carbomer is fully dispersed.
   b. The resulting mixture is homogenized.
   c. The following are added in sequence: EDTA; Tween 20; Preservatives/polyols premix; Organic phase.
   d. Triethanolamine is added till pH=5.5-6.5.
   e. Stirring is continued for 10 min.
4. Active:
   i. Inula helenium extract (100% extract prepared as described in Examples presented herein) is added with stirring to the base mix.
   ii. The resulting mixture is homogenized.

The term “composition for oral care” as used herein refers to a composition suitable for administration to the oral cavity of an individual. The composition can be used as a mouth wash, toothpaste, breath freshener etc. as is known in the art.

The term “inhalation composition” as used herein, refers to a composition directed to the respiratory system. The inhalation composition is designed to reach epithelial cells in the respiratory system.

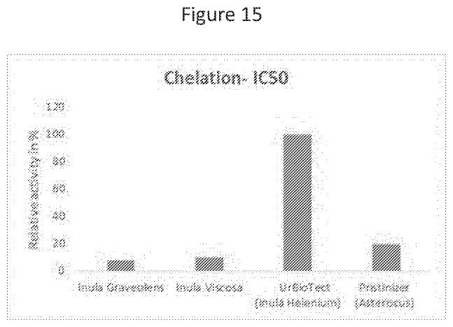

As shown in Table 6 and in FIG. 15, I. helenium extract confers significantly stronger metal-chelating activity than several other Inulae extracts tested to date. The IC50 refers to the amount of extract required to chelate 50% of the available iron (II) in the assay. The lower the number, the higher the chelation power of an extract. Results presented herein demonstrate that I. helenium extract exhibits surprisingly strong metal-chelating activity.

| | Inula | Inula | Inula | Asteriscus |
|---|---|---|---|---|
| Plant | Graveolens | Viscosa | Helenium | Graveolens |
| Fe (ii) Chelation | 1.70 mg/mL | 1.25 mg/mL | 0.13 mg/mL | 0.67 mg/mL |
| power (IC50) | | | | |

### Cosmetic Compositions

The cosmetic compositions of the invention may comprise additional acceptable cosmetic agents as known in the art. According to some embodiments, the cosmetic agent is selected from the group consisting of: botanical extracts, peptides, oligonucletotides, oligo- or poly-saccharides, glycosides, alkaloids, flavonoids, polyphenols, terpenoids, polyketides, carotenoids, fatty acids or derivatives thereof, steroids, xanthines, retinoids, α-hydroxy acids, β-hydroxy acids, α-2 adrenergic inhibitors, β-adrenergic agonists, aromatase inhibitors, anti-estrogens, hydroquinone, ascorbic acid, kojic acid, corticosteroids, mucopolysaccharides, collagen, estrogens, isoflavonoids, cinnamic acid, benzoyl peroxide, tropolone, catechol, mercaptoamine, niacinamide, tocopherol, ferulic acid, azelaic acid, botulinum, urea, a derivative, salt thereof, and any combination thereof. Each possibility represents a separate embodiment of the invention

According to some embodiments, the cosmetic composition further comprises a cosmetically acceptable diluent, carrier or excipient. According to further embodiments, the cosmetically acceptable carrier is selected from the group consisting of a liposome, a micelle structure, a microcapsule, and a combination thereof. Each possibility represents a separate embodiment of the invention.

According to some embodiments, the cosmetic composition further comprises an acceptable additive. According to certain embodiments, the additive is selected from the group consisting of: fats, emulsifiers, co-emulsifiers, hydrophilic gelling agents, lipophilic gelling agents, preservatives, solvents, fragrances, fillers, hydrophilic filters, lipophilic filters, dyestuffs, neutralizers, penetration-enhancing agents, polymers and any combination thereof. Each possibility represents a separate embodiment of the present invention.

The quantities of the various additives are those conventionally used in cosmetic and dermatological preparations as is known to a person skilled in the art.

Non-limiting examples of suitable fats include mineral oils, oils of animal origin (lanolin), synthetic oils (isopropyl myristate, octyldodecyl, isostearyl isostearate, decyl oleate or isopropyl palmitate), silicone oils (cyclomethicone or dimethicone) and fluorinated oils. Fatty alcohol, fatty acids, waxes and gums, notably silicone gums and elastomers can also be used as fats.

Non-limiting examples of suitable emulsifiers and co-emulsifiers include polyglycerol fatty acid esters, sucrose fatty acid esters, sorbitane fatty acid esters, oxyethylene sorbitan fatty acid esters, PEG fatty alcohol ethers, glycerol fatty acid esters, alkyl sulphates, alkyl ether sulphates, alkyl phosphates, alkyl polyglucosides and dimethicone copolyols.

Non-limiting examples of suitable hydrophilic gelling include carboxyvinyl polymers (carbomer), acrylic copolymers such as acrylate/alkylacrylate copolymers, polyacrylamids, polysaccharides such as xanthan gum, guar gum, natural gums such as cellulose gum and derivatives, clays and 2-acrylamido-2-methylpropane acid copolymers.

Non-limiting examples of suitable lipophilic gelling agents include modified clays such as bentones, fatty acid metal salts, hydrophobic silica and ethylcellulose.

Non-limiting examples of suitable fillers include talc, kaolin, mica, serecite, magnesium carbonate, aluminum silicate and organic powders such as nylon.

Non-limiting examples of suitable neutralizers include soda, triethanolamine, aminomethyl propanol and potassium hydroxide.

Non-limiting examples of suitable penetration enhancing agents include alcohols and glycols (ethanol and propylene glycol), ethoxydiglycol, alcohols and fatty acids (oleic acid), fatty acid esters and dimethyl isosorbide.

Non-limiting examples of preservatives include benzoic acid, its salts and esters, sorbic acid and its salts, parabens and their salts, triclosan, imidazolidinyl urea, phenoxyethanol, DMDM hydantoin, diazolidinyl urea and chlorphenesin.

Non-limiting examples of suitable solvents include water, ethanol, glycerin, propylene glycol, butylene glycol, phosphate buffer, saline and sorbitol.

According to one embodiment, the composition comprises a bubbling agent as an additive. A bubbling agent is an agent that emits carbon dioxide gas when contacting liquid with a purpose, for example, to burst a capsule or promote intimate contact of a capsule's content with the surrounding material outside of the capsule.

According to one embodiment, the composition comprises other additives such as those Generally Recognized as Safe (GRAS). GRAS is a United States of America Food and Drug Administration (FDA) designation indicating that a chemical or substance added to food is considered safe, and therefore exempted from the usual Federal Food, Drug, and Cosmetic Act (FFDCA) food additive tolerance requirements. According to some embodiments, the food supplement acceptable excipients comprise excipients in powders and other oral powders, excipients in topical powders, excipients in liquids, excipients in ointments or a combination thereof.

Non-limiting examples of excipients in topical powders include zinc oxide, talc, starch, kaolin, borate powder, zinc stearate, magnesium stearate, magnesium carbonate, precipitated calcium carbonate, bismuth subgallate and potassium aluminum sulfate powder.

Non-limiting examples of excipients in liquids include water, glycerin, propylene glycol, sweet-taste syrup, ethanol, fatty oil, ethylene glycol, polyethylene glycol and sorbitol.

Non-limiting examples of excipients in ointments include hydrophobic or hydrophilic base (including oil-soluble base, water-soluble base and suspended base) prepared by mixing fat, fatty oil, lanoline, Vaseline, glycerin wax, Japan wax, paraffin, paraffin sulphate, resins, higher alcohols, plastics, glycols, water and surfactant.

The quantities of the various additives are those conventionally used in food supplement as is known to a person skilled in the art.

### Inhalation Compositions

The inhalation composition may include any excipient as known in the art, For example, US Application No. 2014/0377355 discloses the use of a combination of two or more poloxamers as optional excipients. Suitable poloxamers may include poloxamer 188 and poloxamer 407. According to some embodiments, the micronized poloxamer composition may include a particle size ranging between about 30 μm and about 70 μm.

The inhalation composition may be obtained in powder form and may be used to fill capsules, which may be later employed for inhalation. According to some embodiments, inhalation composition in powder form may be dissolved employing suitable solvents, such as sterile solution of sodium chloride and water, to obtain inhalation composition in solution form. The inhalation composition of the invention in solution form may be delivered to the respiratory tract using suitable inhalation devices, such as metered-dose inhalers (MDIs), dry powder inhalers, aerosols, syringe, pipette, eyedropper, nebulizers, or any suitable inhalation delivery apparatus.

### Oral Care Composition

Another embodiment of the present invention refers to an oral composition comprising the extract of the invention or a fraction thereof. Oral compositions are intended to be brought into contact with the oral cavity, for example in the form of toothpastes, dental gels, dental creams, mouth washes, sugar-free candies for sucking, oral sprays, dental floss or dental care chewing gums. That is why they are also considered to be dental compositions. The term does not encompass food products dedicated for nutrition.

The oral compositions typically comprise an abrasive system (abrasive or polishing agent), such as e.g. silicas, calcium carbonates, calcium phosphates, aluminium oxides and/or hydroxyapatites, surface-active substances, such as e.g. sodium lauryl sulfate, sodium lauryl sarcosinate and/or cocamidopropyl betaine, moisture-retaining agents, such as e.g. glycerol and/or sorbitol, thickening agents, such as e.g. carboxymethylcellulose, polyethylene glycols, carrageenan and/or Laponite®, sweeteners, such as e.g. saccharin, flavour correctants for unpleasant taste impressions, flavour correctants for further, as a rule not unpleasant taste impressions, flavour-modulating substances (e.g. inositol phosphate, nucleotides, such as guanosine monophosphate, adenosine monophosphate or other substances, such as sodium glutamate or 2-phenoxypropionic acid), cooling active compounds, such as e.g. menthol derivatives (e.g. L-menthyl lactate, L-menthyl alkyl carbonates, menthone ketals, menthanecarboxylic acid amides), 2,2,2-trialkylacetic acid amides (e.g. 2,2-diisopropylpropionic acid methylamide), icilin and icilin derivatives, stabilizers and active compounds, such as e.g. sodium fluoride, sodium monofluorophosphate, tin difluoride, quaternary ammonium fluorides, zinc citrate, zinc sulfate, tin pyrophosphate, tin dichloride, mixtures of various pyrophosphates, triclosan, chlorhexidine, cetylpyridinium chloride, aluminium lactate, potassium citrate, potassium nitrate, potassium chloride, strontium chloride, hydrogen peroxide, aromas, sodium bicarbonate and/or odour correctants.

Formulations or products according to the invention in the form of chewing gums or, in particular, dental care chewing gums comprise chewing gum bases which comprise elastomers, such as, for example, polyvinyl acetates (PVA), polyethylenes, (low or medium molecular weight) polyisobutenes (PIB), polybutadienes, isobutene-isoprene copolymers (butyl rubber), polyvinyl ethyl ethers (PVE), polyvinyl butyl ethers, copolymers of vinyl esters and vinyl ethers, styrene/butadiene copolymers (styrene/butadiene rubber, SBR) or vinyl elastomers, e.g. based on vinyl acetate/vinyl laurate, vinyl acetate/vinyl stearate or ethylene/vinyl acetate, and mixtures of the elastomers mentioned, as described, for example, in EP 0 242 325, U.S. Pat. Nos. 4,518,615; 5,093,136; 5,266,336; 5,601,858 or 6,986,709. In addition, chewing gum bases comprise further constituents, such as, for example, sugars, sugar substitutes or sweet-tasting substances in particular those described in WO 2009/21558, (mineral) fillers, plasticizers, emulsifiers, antioxidants, waxes, fats or fatty oils, such as, for example, hardened (hydrogenated) plant or animal fats, and mono-, di- or triglycerides. Suitable (mineral) fillers are, for example, calcium carbonate, titanium dioxide, silicon dioxide, talc, aluminium oxide, dicalcium phosphate, tricalcium phosphate, magnesium hydroxide and mixtures thereof. Suitable plasticizers or agents for preventing sticking (detackifiers) are, for example, lanolin, stearic acid, sodium stearate, ethyl acetate, diacetin (glycerol diacetate), triacetin (glycerol triacetate) and triethyl citrate. Suitable waxes are, for example, paraffin waxes, candelilla wax, carnauba wax, microcrystalline waxes and polyethylene waxes. Suitable emulsifiers are, for example, phosphatides, such as lecithin, and mono- and diglycerides of fatty acids, e.g. glycerol monostearate.

Formulations or products according to the invention (in particular those which are in the form of an oral care formulation or product or in the form of a formulation) preferably additionally comprise one or more aroma and/or flavouring substances, such as essential oils and extracts, tinctures and balsams, such as, for example, anisole, basil oil, bergamot oil, bitter almond oil, camphor oil, citronella oil, lemon oil; Eucalyptus citriodora oil, eucalyptus oil, fennel oil, grapefruit oil, camomile oil, spearmint oil, caraway oil, lime oil, mandarin oil, nutmeg oil (in particular nutmeg blossom oil=maces oil, mace oil), myrrh oil, clove oil, clove blossom oil, orange oil, oregano oil, parsley (seed) oil, peppermint oil, rosemary oil, sage oil (clary sage, Dalmatian or Spanish sage oil), star aniseed oil, thyme oil, vanilla extract, juniper oil (in particular juniper berry oil), wintergreen oil, cinnamon leaf oil; cinnamon bark oil, and fractions thereof, or constituents isolated therefrom.

The concentration of the Inula plant (e.g., I. helenium) within a selected composition depends on the composition type and intended use. According to certain exemplary embodiments, the composition comprises the extract or the fraction thereof derived from Inula plant at a concentration of 0.1% to 10% (w/w). According to other embodiments, the composition comprises the extract or the fraction thereof at a concentration of 0.2% to 5%. According to certain exemplary embodiments, the composition comprises the extract or the fraction thereof at a concentration of 1% (w/w) relative to the total weight of the composition.

The following examples are presented in order to more fully illustrate some embodiments of the invention. They should, in no way be construed, however, as limiting the broad scope of the invention. One skilled in the art can readily devise many variations and modifications of the principles disclosed herein without departing from the scope of the invention.

### EXAMPLES

### Example 1: Preparation of an Extract of Inula helenium

Dry Inula helenium plant parts (having a moisture content of 10% (w/w) or less) were added to water at 2:1 w/w to 1:30 w/w ratio (plant parts:water) at room temperature. Plants parts were typically dried in an oven with air circulation set to a temperature of 20-60° C. for 12-72 h. The mixture dried plant parts and water was heated for 0.5-6 hours at 20-100° C. and left to cool overnight. Solids were removed by centrifugation at 4500 rpm for 30 min. Next, the supernatant was collected and filtered through 1.2 micron filter, then through 0.45 micron filter and lastly though 0.22 micron filter. The filtrate was collected. To the filtrate, 50-60% Glycerin was added to form the final solution. The formed solution is clear and homogenous, having a pH at the range of 4.0-6.5. The resulting solution is referred to herein as 100% Inula helenium extract.

### Example 2: Identification of Inula helenium Extract as a Protectant Against Environmental Pollution

Environmental pollution is one of the major causes of human skin damage. As described herein, the efficacy of Inula extracts for protecting skin from pollution-associated damage has been substantiated by clinical testing. To advance the understanding of how Inula extracts protect skin from damage due to environmental pollutants, the present inventors developed a sensitive in vitro assay that takes advantage of the susceptibility of Dunaliella salina cell cultures to various toxins/pollutants. Such an in vitro assay also provides a system ideally suited to comparing Inula extracts to assure standardization and quality control among lots made from different batches of Inula plants and made at different times.

In the interests of developing a sensitive, reliable, and cost effective model system for evaluating susceptibility to exogenous toxins, the present inventors investigated Dunaliella salina cells for responsiveness to exogenous toxins. The choice of Dunaliella salina cells was suggested in part because algae are known to be a sensitive model system in which to evaluate water quality in light of their nutritional needs, rapid proliferative rate, and short life cycle. The Dunaliella salina cell culture system was also chosen in part because the cells remain in suspension and therefore, are maximally exposed to toxins introduced into the cell media. Furthermore, since cellular mechanisms relating to susceptibility to the toxic effects of pollutants and for detoxification therefrom are evolutionarily conserved, it was reasonable to expect similar cellular responses in toxicity assays. The utility of the Dunaliella salina cell culture system as a model system for investigating human cell toxin sensitivity is, moreover, underscored by a shared spectrum of sensitivity to toxins with human cells tested to date.

In light of the above, the Dunaliella salina cell culture model was utilized to examine the anti-pollution effect of Inula helenium extracts. Preliminary trials were conducted to evaluate the effective concentration of each toxin. The selected toxins and their concentrations were methanol at 60,000 ppm, gasoline (95 octane gasoline) at 500 ppm and formaldehyde at 25 ppm. Dunaliella cultures were incubated with Inula extract at concentrations of 0% (control), 1% and 2%, for 4 days. After incubation, the cell density (cells/ml) of the culture was evaluated using a digital camera at time point 0 (T0). Each toxin was added to the cultures at T0 and the cultures were maintained under growth promoting conditions and in the presence of Inula extract for another 48 hours. After 48 hours, the cell density (cells/ml) of the cultures was evaluated. Culture growth was calculated as cell density at 48 hours/cell density at T0.

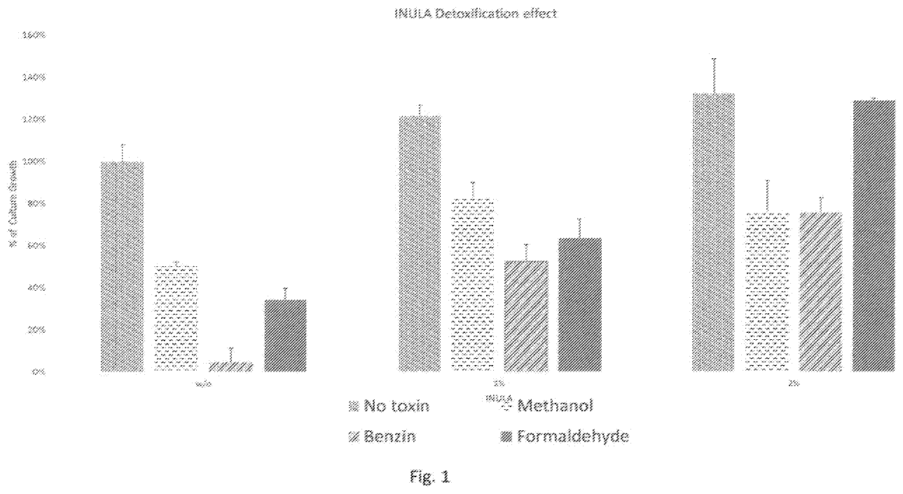

As shown in FIG. 1 and presented in numerical form in Table 2, incubation of Dunaliella salina cell cultures in the presence of Inula extract enhances cell culture growth relative to culturing in the absence of Inula extract. These results demonstrate that Inula extract exhibits a prebiotic effect on the Dunaliella salina cells. Indeed, incubation with 1-2% Inula extract improves culture growth significantly as compared to cultures incubated without Inula extract (negative control: %100±8%; 1% Inula extract: %121±5%; 2% Inula extract: %132±16%). Results depicted in FIG. 1 and Table 2 also reveal that the presence of toxins reduces culture growth relative to cultures incubated in the absence of Inula extract. These results also show that the presence of Inula extract at least partially rescues the Dunaliella salina cell culture from the toxic effects of each of the toxins examined. With respect to formaldehyde mediated toxicity, the detoxification/restorative effects of Inula extract increase with increasing concentration in the cell culture medium. Indeed, the presence of 2% Inula extract in the formaldehyde treated cell cultures restores growth to 129%, which is almost equivalent to culture growth in the presence of 2% Inula extract without toxin (132%). The detoxification/restorative effects of Inula extract are also observed with respect to the toxic effects of methanol as evidenced by an improvement in culture growth in the presence of Inula extract. The level of protection is approximately the same for 1% and 2% Inula extract (%83±7% and %76±14%, respectively). The toxic effects of gasoline are also partially remediated by Inula extract as evidenced by an improvement in culture growth in the presence of added extract. The level of protection increases from %52±8% in the presence of 1% Inula extract to %76±7% in the presence of 2% Inula extract.

Table 3 presents results demonstrating the detoxification effect of Inula extracts.

| | | INULA | | |
|---|---|---|---|---|
| | | w/o | 1% | 2% |
| Toxin | No toxin | 100% ± 8% | 121% ± 5% | 132% ± 16% |
| | Methanol | 50% ± 2% | 83% ± 7% | 76% ± 14% |
| | Gasoline | 5% ± 7% | 52% ± 8% | 76% ± 7% |
| | Formaldehyde | 34% ± 6% | 63% ± 9% | 129% ± 1% |

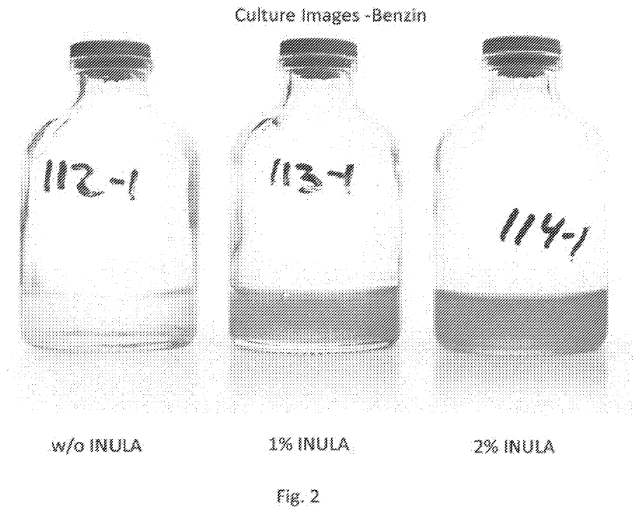

FIG. 2 depicts the protective effects of Inula extract against the toxic effects of gasoline in the Dunaliella salina cell culture system. When examined visually, the green color conferred by live Dunaliella salina cells and opacity associated therewith of the cell culture medium serves as an indicator of cell viability in this system. As shown in the left culture bottle of FIG. 2, wherein Dunaliella salina cells are incubated in the presence of gasoline, without supplemental Inula extract, the cell culture is essentially clear, indicating that few viable cells remain after incubation in the presence of gasoline. In other words, the clarity of the cell culture (lack of opacity) reflects cell death in the presence of the toxin gasoline. As shown in the middle culture bottle of FIG. 2, wherein Dunaliella salina cells are incubated in the presence of gasoline with supplemental Inula extract (1%), the opacity of the cell culture is dramatically increased. The presence of an increased concentration of Inula extract (2%) is associated with an even greater increase in opacity of the cell culture. These results demonstrate that gasoline-related toxicity of Dunaliella salina cell culture growth can be at least partially rescued by the presence of supplemental Inula extract, and increasing concentrations of Inula extract are positively correlated with increasing cell viability in the presence of gasoline.

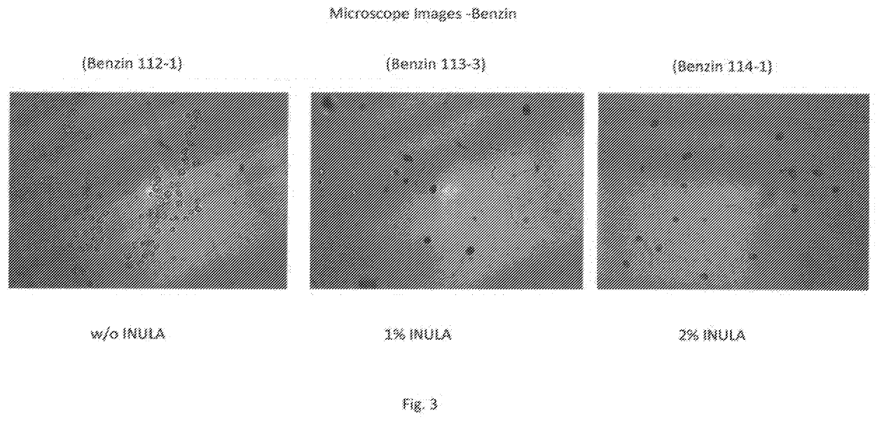

The ability of Inula extract to remediate the toxic effects of gasoline on Dunaliella salina cells is depicted in microscope images in FIG. 3. As shown in the left panel of FIG. 3, most of the cells are dead and therefore appear transparent when incubated in the presence of gasoline and visualized microscopically. The presence of 1% Inula extract rescues a significant percent of Dunaliella salina cells as reflected by the presence of live cells that appear opaque when visualized microscopically. The number of live cells evident in Dunaliella salina cell cultures incubated in the presence of 2% Inula extract is further increased relative to that seen in the presence of 1% Inula extract. These results indicate that there is a dose dependent response between increased cell viability and increased amounts of Inula extract.

### Example 3: Identification of Inula helenium Extract as a Protectant Against the Toxic Effects of Heavy Metals

The ability of Inula extract to act as an anti-pollutant or antagonist of the adverse effects of pollutants and/or toxins in pollutants was further investigated in heavy metal chelation assays. Further to this objective, Inula extracts was tested in in vitro assay to assess its chelator power. In this assay, the ferrous ion is used as a model for heavy metals. The concentration of the ferrous iron-Ferrozine complex was measured by spectrophotometry (absorbance at 562 nm) to determine the Fe 2+-chelating ability of the extract or fraction. The reaction mixture, containing 2 Mm FeCl2 and 5 mM ferrozine was adjusted to 2 ml with bi-distilled water. The mixture was shaken and incubated for 10 min at room temperature. The absorbance of the mixture was measured at 562 nm against a blank EDTA was used as positive control. The choice of an internal reference may be adjusted in accordance with the heavy metal assay performed and such references are known in the art. Chelating power is expressed as an IC50 value (mg/ml) and reflects the concentration at which 50% of the available iron was chelated by, for example, supplemental extract added to the assay mix.

Table 4 presents results demonstrating chelating properties of Inula extracts.

| Sample | IC50* (mg/ml DRY WEIGHT) |
|---|---|
| DC1-4-2 | 0.46 |
| DC1-25B | 0.67 |
| DC1-18-0 | 0.50 |
| AVERAGE | 0.54 |
| EDTA (reference) | 0.038 |

*calculated based on unpreserved extract dry weight

Results relating to the chelating properties of Inula extract in heavy metal chelation assays are presented in Table 3. Results presented in Table 3 reveal that Inula extracts possess significant chelating power. More particularly, Inula extracts tested to date exhibit approximately one (1) order of magnitude lower chelating power relative to that of the reference chelator EDTA on a dry weight basis. It is noteworthy in this regard that EDTA is used in the assay as a pure sample and is known to be a powerful chelator. Therefore, the results presented with respect to the chelating properties of Inula extracts are remarkable given that the extract is being tested as an Inula-derived extract comprising a variety of Inula plant agents/compounds in solution.

### Example 4: Clinical Evaluation of Inula helenium Extract as an Antagonist of Pollutant-Mediated Damage to Skin

Cigarette smoke was chosen as a model pollutant because it is common environmental contaminant with proven adverse effects on health in general and skin health and appearance in particular. Cigarette smoke comprises many toxic compounds, including volatile organic compounds (VOCs), polyaromatic hydrocarbons (PHAs), oxides, and heavy metals. Specific examples of pollutants known to be present in cigarette smoke that have adverse effects on epithelial cells include formaldehyde, methanol, and polyaromatic hydrocarbons.

A clinical study was performed to evaluate Inula extract's activity as a ‘detoxifying’ agent in vivo. Based on results presented herein, Inula extracts possess properties that confer, upon application to skin, global anti-aging benefits resulting from enhanced resistance of the skin to chronic toxic insult.

Study Design:
Double blind test: formulation comprising 1% I. helenium extract vs. placebo (cream-gel chassis only).
Age distribution: 46-60; Group size=26; all-female.
Phototype (Fitzpatrick) II to III
All skin types
All subjects have a heavy smoking habit (a minimum of 10 cigarettes/day) and have ageing signs (wrinkles or small riddles and yellowish skin) in the periocular area.
Randomized split-face (left/right) application twice daily for 28 days, with evaluation at D0, D14, and D28. Effects of chronic exposure to cigarette smoke and toxins therein are evident on the placebo-treated side of a subject, whereas restorative effects due to topical administration of formulations comprising 1% I. helenium extract are evaluated on the extract-treated side of a subject.

Measurements include:
Visia CA: multiple parameters were measured, including wrinkles and spots using standardized imaging with normal, cross-polarized, and UV illumination.
Primos 3D: used to evaluate wrinkles and roughness.
Chromameter: used to evaluate skin color (as L, a, b; using, e.g., a D65 illuminant).
Cutometer: used to evaluate skin firmness and elasticity.
Tewameter: used to evaluate skin barrier properties (TEWL).
Visioscan: used to evaluate skin homogeneity
Vivascope confocal microscopy: used to evaluate skin morphology, particularly at the dermal-epidermal junction
Clinical assessment by a dermatologist: used to evaluate, e.g., wrinkles, evenness of complexion, and lightening effect.
Subjective self-assessment by questionnaire: used to evaluate subject's disposition toward anti-pollutant regimen which reflects subject's perception of physical manifestations of exposure to cigarette smoke borne pollutants and potential protection therefrom conferred by administration of formulations described herein.

Table 5 presents the formulations tested on the skin of subjects, which are as follows:

| | | % in formula - | % in formula - |
|---|---|---|---|
| | | ACTIVE | PLACEBO |
| INCI/chemical name | Tradename | IBRD1758 | IBRD1759 |
| Water | Water | 84.35 | 84.85 |
| Butylene glycol | 1,3-butanediol | 4.00 | 4.00 |
| Dipropylene glycol | Dipropylene | 1.00 | 1.00 |
| | glycol | | |
| Hexylene glycol | Hexylene glycol | 1.00 | 1.00 |
| Polysorbate 20 | Tween 20 | 1.00 | 1.00 |
| Hydrogenated polydecene | Nexbase | 1.50 | 1.50 |
| Cyclomethicone | SF0005Z | 4.00 | 4.00 |
| Inula Helenium extract | Inula Helenium | 1.00 | 0.00 |
| | extract | | |
| Glycerol | Glycerin | 0.00 | 0.50 |
| Carbomer | Carbopol 940 | 0.80 | 0.80 |
| Triethanolamine | Triethanolamine | 0.70 | 0.70 |
| Phenoxyethanol | Phenoxyethanol | 0.40 | 0.40 |
| Methyl paraben | Nipagin M | 0.15 | 0.15 |
| EDTA | EDTA | 0.10 | 0.10 |

| | | |
|---|---|---|
| TOTAL | 100.00 | 100.00 |

### Results:

### Skin Color (Chromameter):

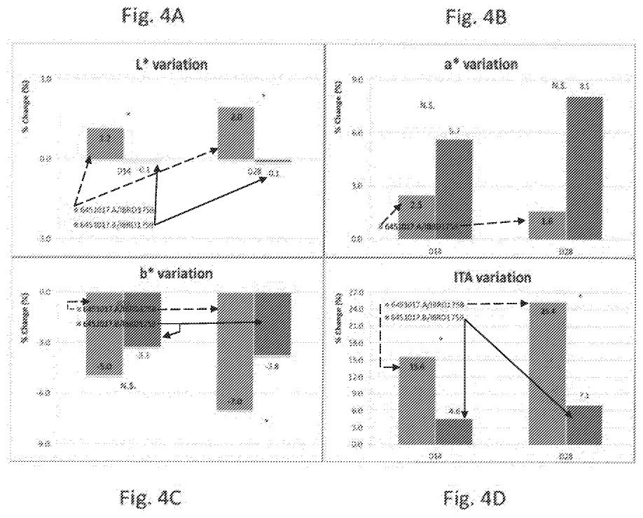

Topical application of a formulation comprising an Inula plant (e.g., Inula helenium) extract described herein resulted in a small, but significant lightening effect on skin as compared to skin measured at day 0 (baseline levels) or skin measured following topical application of placebo at comparative times. The lightening effect was manifest as an increase in L* (i.e., brighter skin). See FIG. 4A. A significant decrease in sallowness b* (i.e., reduction in yellow skin) was also observed relative to baseline levels (measured at day 0) or levels observed following topical application of placebo at comparative times. See FIG. 4C. The Individual Typological Angle (ITA), calculated based on L* and b* measurements, also shows a significant increase in skin lightening associated with application of Inula plant extract-containing formulations as compared to skin color measured at day 0 (baseline levels) or skin color measured following topical application of placebo at comparative times. See FIG. 4D.

Without wishing to be bound by any specific theory or mechanism of action, this effect may be due to a detoxification mechanism. Skin that is chronically challenged by pollutants has a tendency to produce elevated melanin levels, which typically results in elevated or uneven skin pigmentation, spots, and other discolorations. Exposure to pollutants present in cigarette smoke creates a condition of chronic, low-grade inflammation in the skin of persons subjected to the smoke, either via first or second hand exposure. Extracts and/or compositions or formulations thereof described herein provide protection against pollutant-driven adverse effects such as, for example, chronic, low-grade inflammation and/or elevated or uneven skin pigmentation. The protective effects of Inula extracts and/or compositions or formulations thereof described herein are apparent as early as day 14 post-administration and these effects continue to increase up until at least day 28 post-administration. These results are consistent with an effect that requires time for cell turnover. Some degree of desquamation, for example, may be necessary to visualize the beneficial effects of administration of Inula extracts and/or compositions or formulations thereof to skin under chronic stress due to habitual exposure to pollutants. Cell turnover via desquamation, whereby existing melanin-loaded keratinocytes/corneocytes are replaced by new cells, produces a new generation of cells which mature under the influence of Inula extract and thus experience lower levels of chronic toxic stress.

### Anti-Aging (Anti-Wrinkles):

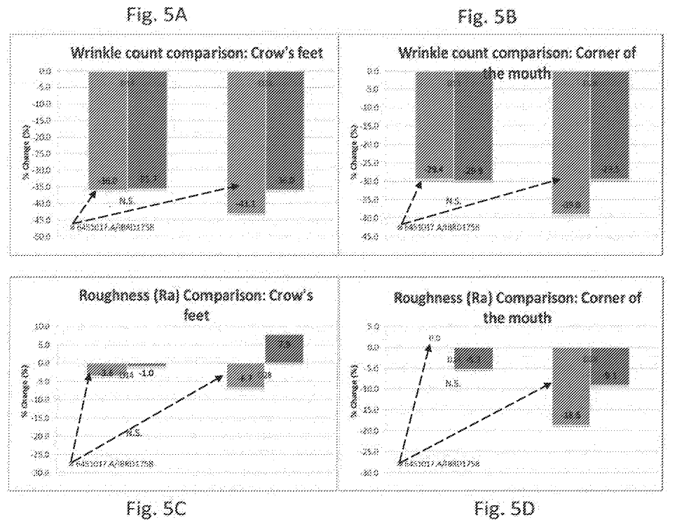

Inula extracts and/or compositions or formulations thereof exhibit good anti-aging properties, as manifest by a strong and significant reduction in wrinkle count at both the corner of the eye and the corner of the mouth by day 28 post-administration. See FIGS. 5A and 5B. Indeed, the wrinkle count was reduced by about 40% at day 28 as compared to day 0 (baseline levels). Although administration of placebo also registered a decrease in wrinkle count by day 28 post-administration, the decrease observed following treatment with formulations comprising Inula extract is statistically significant relative to that of placebo. A significant improvement in skin roughness was also observed relative to skin roughness determined at day 0 (baseline levels) or in placebo treated skin at day 28 post-administration. The improvement in Inula extract formulation-treated skin was particularly pronounced at the corner of the mouth (about a 20% decrease in roughness was detected).

### Clinical Evaluation:

### Wrinkles:

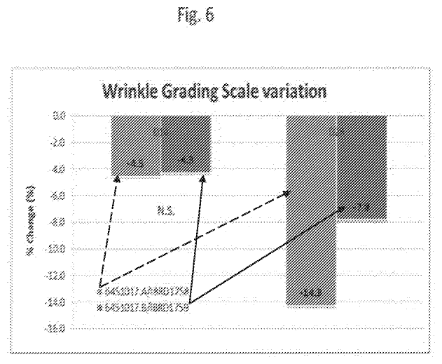

To investigate further the instrumental measurements determined using Primos 3D, clinical evaluations were performed. These evaluations revealed a significant anti-aging effect, expressed as a significant reduction in wrinkle score at the corner of the eye relative to day 0 (baseline) and placebo. See FIG. 6.

### Other Significant Clinical Parameters:

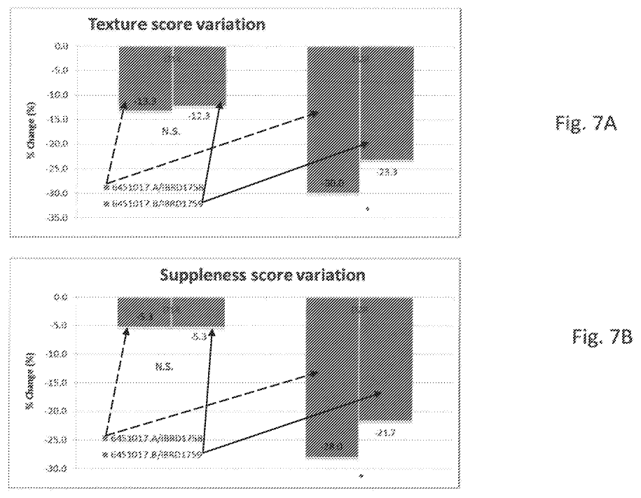

Formulations comprising Inula plant (e.g., Inula helenium) extract also conferred pollutant-protecting properties as evidenced by a significant improvement in skin texture and skin suppleness grades by day 28 post-administration as compared to either day 0 (baseline) or placebo at day 28 post-administration. See FIGS. 7A and 7B.

### Visia Results:

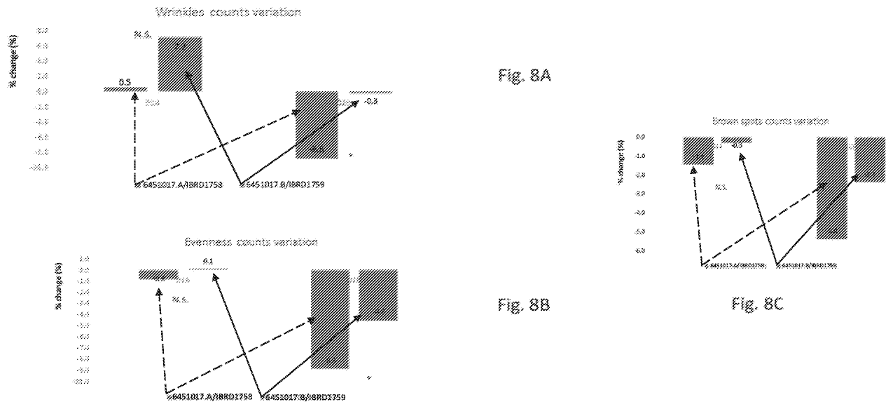

Formulations comprising Inula plant (e.g., Inula helenium) extract also conferred the following pollutant-protecting properties:
A modest but significant effect on wrinkle counts by day 28 post-administration as compared to either day 0 or placebo at day 28 post-administration. See FIG. 8A. These results corroborate results generated using the Primos system.
A modest but significant improvement in skin evenness by day 28 post-administration as compared to either day 0 or placebo at day 28 post-administration. See FIG. 8B.
A small but significant improvement in brown spot counts by day 28 post-administration as compared to either day 0 or placebo at day 28 post-administration. See FIG. 8C. Brown spot area is also improved by administration of Inula plant extract formulations, particularly with respect to that observed following administration of placebo formulations, which exhibit enlargement of brown spot area at day 28 post-administration.
A small, but significant, improvement in visible spot counts was also detected as compared to day 0.

### Skin Barrier (TEWL):

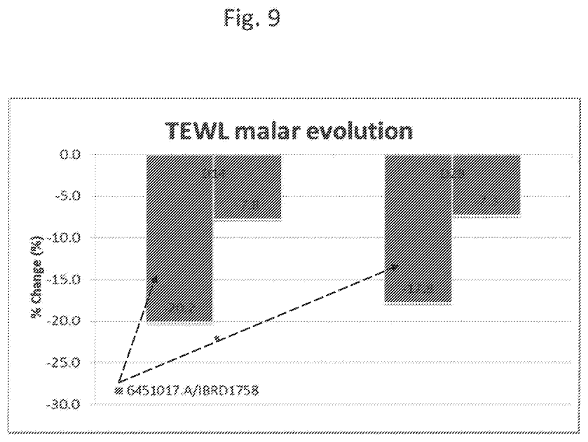

Formulations comprising Inula plant (e.g., Inula helenium) extract also conferred a significant improvement in TEWL malar evolution relative to day 0 (baseline) or placebo at either day 14 or day 28 post-administration. See FIG. 9. FIG. 9 also reveals that formulations comprising Inula plant extract confer benefit rapidly, as they are evident by day 14 post-administration. Given the turnover rate of cells in the skin, the rapid response rate reveals a surprising property of Inula extracts to combat adverse effects of pollutants rapidly.

### Skin Texture (Visioscan):

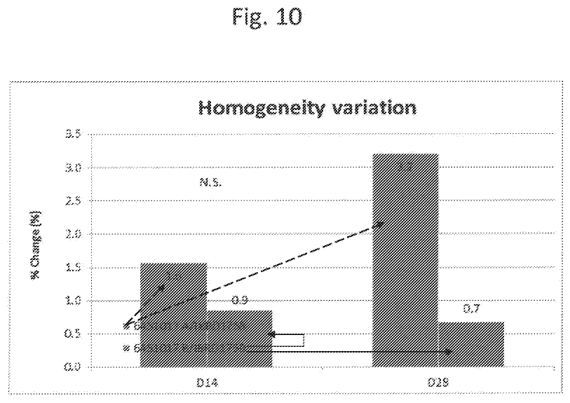

Formulations comprising Inula plant (e.g., Inula helenium) extract also conferred a small, but statistically significant improvement in skin homogeneity relative to day 0 (baseline) or placebo at day 28 post-administration. See FIG. 10.

### Self-Evaluation Questionnaire:

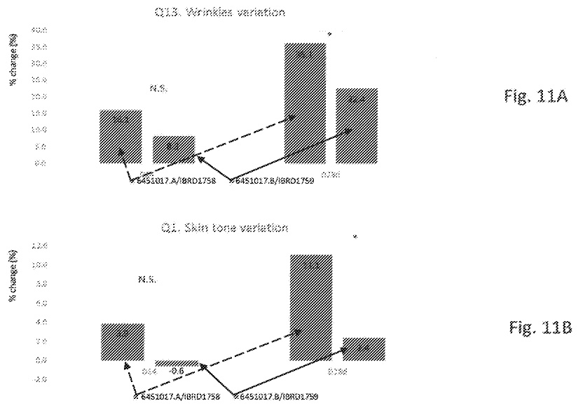

The Inula helenium plant extract described herein and formulations and compositions comprising the extract further improved skin parameters such as skin tone variation and wrinkles in a statistically significant fashion at day 28 post-administration as reflected by self assessment of subjects participating in the study. See FIGS. 11A and 11B. The statistical significance of these improvements were apparent when compared to either day 0 (baseline) or placebo at 28 day post-administration.

### Skin Elasticity

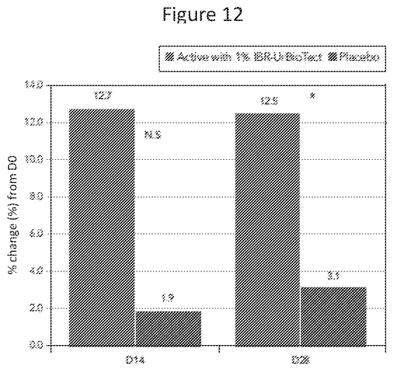

Formulations comprising Inula plant (e.g., Inula helenium) extract also improved skin elasticity around the eye (periorbital) in a smoking environment. Cutometer data shows that daily application of Inula helenium extract results in a meaningful improvement in skin elasticity after 28 days, statistically significant vs. day 0 (D0) and vs. the placebo. The effect is shown to build up over the course of the study. The improvement was already visible, but had not yet acquired statistical significance at D14 of application regimen. See FIG. 12.

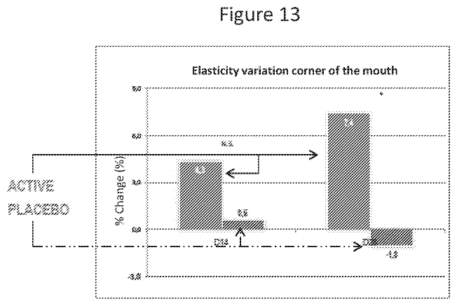

Formulations comprising Inula plant (e.g., Inula helenium) extract also improved skin elasticity around the corner of the mouth in a smoking environment. Cutometer data shows that daily application of Inula helenium extract results in a meaningful improvement in skin elasticity after 28 days, statistically significant vs. day 0 (D0) and vs. the placebo. The effect is shown to build up over the course of the study. The improvement was already visible, but had not yet acquired statistical significance at D14 of application regimen. See FIG. 13.

Protection from Blue Light-HEV

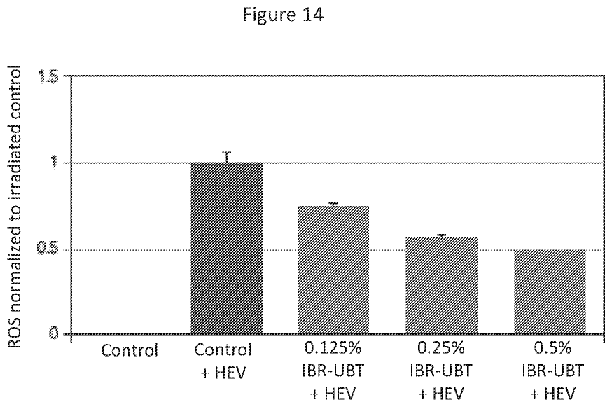

In order to explore additional pollution-protective effects of Inula helenium extract, experiments to evaluate the adverse effects of blue Light (HEV) on human skin cells were performed in the presence and absence of Inula helenium extract. As shown in FIG. 14, Inula helenium extract efficiently protected skin cells from HEV induced ROS production. These results demonstrate that Inula helenium extract exhibits protection against a diverse range of environmental pollution, as demonstrated by significant protective benefits conferred by application of the extract to skin cells exposed to environmental pollution.

### General Comment on Time-Dependency:

Although a trend toward realization of beneficial effects may be suggested by day 14, acquiring results having statistical significance frequently requires a 28 day incubation period in the presence of Inula extract or a formulation or composition thereof. Without wishing to be bound by any specific theory or mechanism of action, these results support a repair mechanism induced by Inula helenium, whereby cell intrinsic, active processes induced which confer cellular recovery from adverse effects of chronic toxic stress on the skin. When cellular recovery spreads to a discernable or detectable level (e.g., using a suitable machine or by visual evaluation by a clinician or other person), the skin re-establishes a healthy state of homeostasis, which in turn makes skin more radiant and beautiful.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1: A method for protecting a tissue comprising epithelial cells from adverse effects of at least one pollutant, the method comprising administering to a subject in need thereof at least one of:
(a) a polar solvent extract derived from an Inula plant,
(b) a fraction of the polar solvent extract derived from an Inula plant, or
(c) a composition comprising the polar solvent extract derived from an Inula plant or the fraction of the polar solvent extract derived from an Inula plant,
in an effective amount sufficient to protect the tissue comprising epithelial cells from adverse effects of the at least one pollutant, thereby protecting the tissue from the at least one pollutant.

2: The method of claim 1, wherein the administering is performed before, during, or after exposure to the at least one pollutant.

3-9. (canceled)

10: The method of claim 1, wherein the tissue is skin and wherein the method comprises topically administering at least one of the polar solvent extract, the fraction of the polar solvent, and the composition comprising the polar solvent extract or the fraction thereof to the skin.

11-22. (canceled)

23: The method of claim 1, wherein the polar solvent is water.

24: The method of claim 1, wherein the polar solvent comprises water and at least one additional polar solvent selected from the group of glycerin, ethanol, propylene glycol, butylene glycol, methanol and acetone.

25. (canceled)

26: The method of claim 24, wherein the polar solvent comprises water and glycerin.

27: The method of claim 1, wherein the polar solvent extract or the fraction of the polar solvent extract is essentially devoid of essential oils.

28: The method of claim 1, wherein the polar solvent extract or the fraction of the polar solvent extract is derived from the Inula plant, excluding roots of the Inula plant.

29: The method of claim 1, wherein the Inula plant is Inula helenium.

30: A composition comprising a polar solvent extract derived from an Inula plant or a fraction of the polar solvent extract derived from an Inula plant in:
(a) an amount effective to protect a tissue comprising epithelial cells from adverse effects of at least one pollutant, or
(b) an effective amount sufficient to at least partially restore tissue function,
the composition optionally further comprising a cosmetically, pharmaceutically, or dermatologically acceptable diluent, excipient or carrier.

31: The composition according to claim 30, wherein the Inula plant is Inula helenium.

32: The composition according to claim 30, wherein the polar solvent extract or the fraction of the polar solvent extract is derived from the whole Inula plant or any aerial parts of the Inula plant.

33-34. (canceled)

35: The composition of claim 30, wherein the polar solvent is water.

36: The composition of claim 30, wherein the polar solvent comprises water and at least one additional polar solvent selected from the group of glycerin, ethanol, propylene glycol, butylene glycol, methanol and acetone.

37. (canceled)

38: The composition of claim 30, wherein the polar solvent extract or the fraction of the polar solvent extract comprises up to 1% w/w essential oils.

39. (canceled)

40: The composition of claim 30, further comprising at least one additional plant extract or at least one microalgae extract.

41. (canceled)

42: The composition of claim 30, further comprising an additional active agent comprising an anti-oxidant, a chelator, a cleansing agent, a skin protectant, a sunscreen, a skin lightening agent, an anti-wrinkling agent, an anti-inflammatory agent, an anti-aging agent, or any combination thereof.

43-58. (canceled)

59: A polar solvent extract derived from an Inula plant/or a fraction of the polar solvent extract or a composition comprising a polar solvent extract derived from an Inula plant/or a fraction of the polar solvent extract for use in protecting a tissue comprising epithelial cells from adverse effects of at least one pollutant or at least partially restoring tissue function of a tissue comprising epithelial cells damaged by exposure to adverse effects of at least one pollutant.

60: The polar solvent extract of claim 59, wherein the Inula plant is Inula helenium.

61: The polar solvent extract of claim 59, wherein the polar solvent extract or the fraction of the polar solvent extract is derived from the Inula plant, excluding roots of the Inula plant.

62-64. (canceled)
